(19) United States
(12) Patent Application Publication
(10) Pub. No.: US 2010/0030216 A1
(43) Pub. Date: Feb. 4, 2010

(54) DISCECTOMY TOOL HAVING COUNTER-ROTATING NUCLEUS DISRUPTORS

(76) Inventor: Gregory B. ARCENIO, Redwood City, CA (US)

Correspondence Address: Gregory B. Arcenio, 220 Oak Avenue, redwood City, CA, 94061, US

(21) Appl. No.: 12/182,422

(22) Filed: Jul. 30, 2008

(51) Int. Cl. A61B 17/00; A61F 5/04; A61B 17/32

(52) U.S. Cl. 606/79; 606/53; 606/170

(57) ABSTRACT

Spinal tools and methods are described herein. In some embodiments, an apparatus includes an elongate member and a tissue disrupter. The elongate member has a distal end portion and defines a lumen. The tissue disruptor is coupled to the distal end portion of the elongate member. The tissue disruptor is configured to rotate relative to the elongate member to disrupt a body tissue.

### BACKGROUND

The invention relates generally to the treatment of spinal conditions, and more particularly, to tools and methods used to remove at least a portion of the nucleus of an intervertebral disc.

Tools and procedures have been developed to remove the nucleus of an intervertebral disc in preparation for nucleus replacement therapy or interbody fusion. Known rongeurs are used to remove the nucleus of the intervertebral disc. To perform a discectomy and/or nucleus removal using one or more rongeurs, a medical practitioner creates a sizable opening in the patient's body and in the annulus of the intervertebral disc. The medical practitioner then repeatedly inserts and withdraws the one or more rongeurs from the patient's body. This repeated insertion and removal, however, can cause trauma and/or damage to the patient's body. Additionally, nucleus removal can take a significant amount of time because the rongeur is repeatedly inserted and withdrawn from the patients body. Further, removal of the entire nucleus of the intervertebral disc using a rongeur is difficult because direct visualization is used to determine where the remaining portion of the nucleus is disposed within the intervertebral disc.

Thus, a need exists for improvements in the tools and procedures used to remove at least a portion of the nucleus of an intervertebral disc. Specifically, tools and procedures are needed to perform minimally-invasive removal of at least a portion of the nucleus of an intervertebral disc. Additionally, tools and procedures are needed to reduce the amount of time it takes to remove the nucleus of an intervertebral disc.

### SUMMARY

Spinal tools and methods are described herein. In some embodiments, an apparatus includes an elongate member and a tissue disruptor. The elongate member has a distal end portion and defines a lumen. The tissue disruptor is coupled to the distal end portion of the elongate member. The tissue disrupter is configured to rotate relative to the elongate member to disrupt a body tissue.

### BRIEF DESCRIPTION OF THE DRAWINGS

FIG. 1 is a schematic illustration of a medical tool, according to an embodiment.

FIG. 2 is a schematic illustration of a medical tool, according to an embodiment.

FIGS. 3 and 4 are schematic illustrations of a medical tool in a first configuration and a second configuration, respectively, according to an embodiment.

FIG. 5 is a schematic illustration of a medical tool, according to an embodiment.

FIG. 6 is a perspective view of a medical tool, according to an embodiment.

FIGS. 7 and 8 are close-up views of a distal end portion of the medical tool shown in FIG. 6 in a first configuration and a second configuration, respectively.

FIG. 9 is a cross-sectional view of the portion of the medical tool shown in FIG. 6, in the first configuration, taken along line X-X in FIG. 7.

FIG. 10 is a cross-sectional view of the medical tool shown in FIG. 6, in the first configuration, taken along line Y-Y in FIG. 7.

FIG. 11 is a perspective view of a medical tool, according to an embodiment.

FIG. 12 is a cross-sectional view of a portion of the medical tool shown in FIG. 11, taken along line Z-Z in FIG. 11.

FIG. 13 is a cross-sectional view of a portion of the medical tool shown in FIG. 11, in the first configuration, taken along line Z-Z in FIG. 11.

FIG. 14 is a cross-sectional view of a portion of the medical tool shown in FIG. 11, in the second configuration, taken along line Z-Z in FIG. 11.

FIG. 15 is a front perspective view of the medical tool shown in FIG. 11 with the distal cap removed.

FIGS. 16 and 17 are schematic illustrations of a medical tool in a first configuration and a second configuration, respectively, according to an embodiment.

FIGS. 18 and 19 are schematic illustrations of a medical tool in a first configuration and a second configuration, respectively, according to an embodiment.

FIGS. 20 and 21 are schematic illustrations of a medical tool in a first configuration and a second configuration, respectively, according to an embodiment.

FIG. 22 is a flow chart illustrating a method of using a medical tool, according to an embodiment.

### DETAILED DESCRIPTION

In some embodiments, a medical tool includes an elongate member and a tissue disrupter. The elongate member has a distal end portion and defines a lumen. The tissue disrupter is coupled to the distal end portion of the elongate member such that longitudinal movement of the tissue disrupter relative to the elongate member along a center line of the tissue disrupter is limited. The tissue disrupter is configured to rotate relative to the elongate member. The tissue disrupter can cleave, stir, disrupt, and/or sever tissue when disposed within a body of a patient. At least a portion of the tissue disrupter is disposed within the lumen defined by the elongate member. Tissue can be collected within the elongate member when the tissue is cleaved, stirred, disrupted, and/or severed by the tissue disrupter. The center line of the tissue disrupter is offset from a center line of the lumen defined by the elongate member.

In some embodiments, a medical tool includes an elongate member, a first tissue disruptor, and a second tissue disrupter. The elongate member has a distal end portion and defines a lumen. The first tissue disrupter and the second tissue disrupter are coupled to the distal end portion of the elongate member. At least a portion of the first tissue disrupter and at least a portion of the second tissue disrupter are disposed within the lumen. The first tissue disrupter is configured to rotate relative to the elongate member in a first direction. The second tissue disrupter is configured to rotate relative to the elongate member in a second direction, opposite the first direction. In this manner, tissue can be cleaved, stirred, disrupted, and/or severed by the first tissue disrupter and the second tissue disruptor.

In some embodiments, a medical tool includes an elongate member and a tissue disrupter. The elongate member has a distal end portion and defines a lumen. The tissue disruptor is coupled to the distal end portion of the elongate member and includes a carriage and a rotatable member. The carriage is rotatably coupled to the distal end portion of the elongate member and is configured to be moved between a first position and a second position. The rotatable member is coupled to the carriage and is configured to rotate relative to the carriage. The rotatable member has a cutting surface configured to be disposed within the lumen of the elongate member when the carriage is in the first position. With the cutting surface disposed within the lumen of the elongate member, the tissue disruptor can be inserted into a body of a patient without damaging surrounding tissue. Once within the body of the patient, the carriage can be moved from its first position to its second position. In the second position, at least a portion of the cutting surface is configured to be disposed outside of the lumen defined by the elongate member. With the cutting surface disposed outside of the lumen defined by the elongate member, tissue can be cleaved, stirred, disrupted, and/or severed by the tissue disrupter.

In some embodiments, an apparatus includes an elongate member, a tissue disruptor, and a threaded member. The elongate member includes a distal end portion and defines a lumen. The tissue disruptor is coupled to the distal end portion of the elongate member and is configured to convey a tissue from a region outside of the elongate member into a distal portion of the lumen. The tissue disruptor is configured to rotate relative to the elongate member. The threaded member is rotatably disposed within the lumen of the elongate member. The threaded member is configured to rotate within the lumen defined by the elongate member. As the threaded member rotates, the threads of the threaded member convey the tissue from the distal portion of the lumen to a proximal portion of the lumen. In this manner, tissue can be removed from a body of a patient.

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to direction closer to and away from, respectively, an operator (e.g., surgeon, physician, nurse, technician, etc.) who would insert the medical tool into the patient. Thus, for example, the end of the medical tool first inserted inside the patient's body would be the distal end of the medical tool, while the end of the medical tool to last enter the patient's body would be the proximal end of the medical tool.

It should be understood that the references to geometric constructions are for purposes of discussion and illustration. The actual structures may differ from geometric ideal due to tolerances and/or other minor deviations from the geometric ideal.

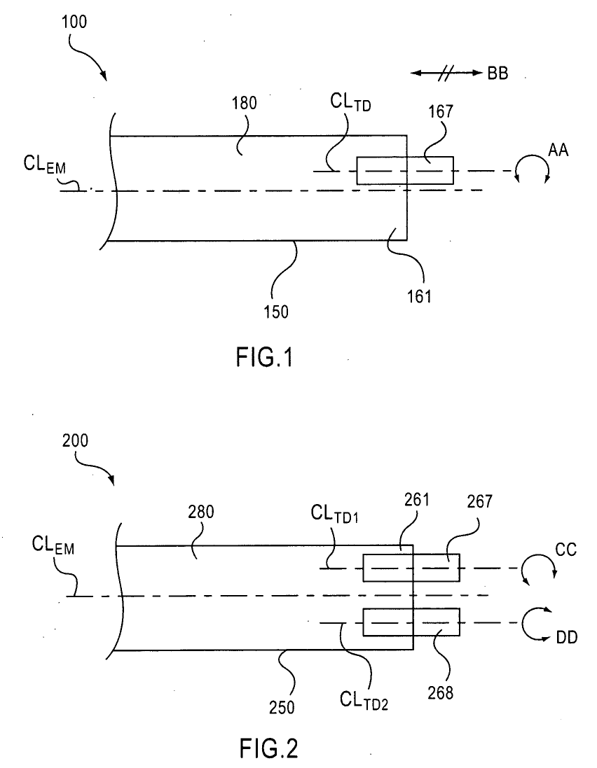

FIG. 1 is a schematic illustration of a medical tool 100, according to an embodiment. Medical tool 100 includes an elongate member 150 and a tissue disrupter 167. The elongate member 150 has a distal end portion 161 and defines a lumen 180. The distal end portion 161 is configured to be inserted into a body of a patient, as further described herein. The lumen 180 defined by the elongate member 150 defines a center line CL_{LEM}. In some embodiments the lumen 180 can be configured to receive tissue of a patient, as further described herein.

The tissue disrupter 167 of the medical tool 100 is coupled to the distal end portion 161 of the elongate member 150 such that movement of the tissue disrupter 167 relative to the elongate member 150 in the direction shown by arrow BB in FIG. 1 is limited and/or prohibited. At least a portion of the tissue disruptor 167 is disposed within the lumen 180. The tissue disruptor 167 is configured to rotate with respect to the elongate member 150 in the direction shown by arrow AA in FIG. 1. In this manner, the tissue disruptor 167 can disrupt body tissue, as described in more detail herein.

The tissue disruptor 167 defines a center line CL_{TD} that is offset from the center line CL_{EM} of the lumen 180 of the elongate member 150. The center line CL_{TD} of the tissue disrupter 167 is substantially parallel to the center line CL_{EM} of the lumen 180 of the elongate member 150. In other embodiments, the center line CL_{TD} of the tissue disrupter can be collinear with the center line CL_{EM} of the lumen of the elongate member and/or the tissue disrupter can be positioned such that the center line CL_{TD} of the tissue disrupter intersects the center line CL_{EM} of the lumen of the elongate member. In still other embodiments, the tissue disrupter can be movable between a first position where the center line CL_{TD} of the tissue disruptor is parallel to the center line CL_{EM} of the lumen of the elongate member and a second position where the center line CL_{TD} of the tissue disrupter intersects the center line CL_{EM} of the lumen of the elongate member.

The tissue disrupter 167 can be substantially rigid. Said another way, the tissue disrupter 167 does not substantially deform when rotated within a body of a patient. In alternate embodiments, the tissue disrupter can be configured to flex and/or bend. Further, while not shown in FIG. 1, the tissue disrupter 167 can have a sharp cutting surface for example, a sharp worm gear, a helical flute, and/or claws. Such a sharp cutting surface can aid the tissue disrupter 167 in disrupting the body tissue when the tissue disrupter 167 of the medical tool is inserted into a body of a patient, as described in further detail below.

In use, the medical tool 100 is inserted into a body of a patient. For example, a medical practitioner can insert the medical tool 100 percutaneously through a cannula into a body of a patient. In one example, the medical tool 100 can be used to treat a herniated intervertebral disc. The medical tool 100 can be inserted into the interior of an intervertebral disc using a method similar to the method described in U.S. application Ser. No. 12/109,565 filed on Apr. 25, 2008 and entitled “Medical Device with One-Way Rotary Drive Mechanism,” which is incorporated herein by reference in its entirety. For example, the medical tool 100 can be used to disrupt and remove nucleus material from an interior of an intervertebral disc. An access path into the intervertebral disc can be made, for example, with a stylet or other access tool through, for example, Kambin's triangle. An optional access cannula can be inserted into an intervertebral disc via the access path. The access cannula is inserted through the annulus of the intervertebral disc and its distal end is disposed within the nucleus of the intervertebral disc (e.g., just inside the annular wall). The medical tool 100 can then be inserted through a lumen of the access cannula and into the nucleus of the intervertebral disc.

Another example of a device that can be used to gain access to an intervertebral disc is described in U.S. patent application Ser. No. 11/250,617, filed Oct. 17, 2005, and entitled “Balloon Assisted Apparatus and Method for Accessing an Intervertebral Disc” (“the '617 application”), which is incorporated herein by reference in its entirety. As described in the '617 application, a device having a sharp tip and a balloon coupled thereto can be inserted through a lumen of a cannula with the balloon in a collapsed configuration. The sharp tip can penetrate the annular wall and the device can be positioned such that the balloon is disposed within the annulus material of the intervertebral disc. The balloon can then be expanded such that the annulus material is distracted by the balloon forming an access opening through the annular wall sufficient to insert the cannula.

Other example procedures to gain access to an intervertebral disc are described in U.S. patent application Ser. No. 10/825,961, filed Apr. 16, 2004, and entitled “Spinal Diagnostic Methods and Apparatus” (“the '961 application”), which is incorporated herein by reference in its entirety. For example, in one embodiment of the '961 application, an introducer device and a pointed obturator are inserted into an intervertebral disc. The pointed obturator is used to penetrate the annular wall of the intervertebral disc and then removed. A guide wire is then inserted through the introducer and used to guide a cannula through the introducer and into the intervertebral disc. In another example described in the '961 application, a catheter having a stylet is passed through an introducer device and into an intervertebral disc without the use of a guide wire.

Once the tissue disruptor 167 of the medical tool 100 is positioned within the body of the patient, the tissue disrupter 167 is rotated with respect to the elongate member 150 in the direction shown by the arrow AA in FIG. 1. By rotating the tissue disruptor 167 in the direction shown by the arrow AA in FIG. 1, the body tissue adjacent the tissue disruptor is cleaved, stirred, disrupted, and/or severed. For example, the tissue disruptor 167 can cleave, stir, disrupt, and/or sever at least a portion of the nucleus of an intervertebral disc when the medical tool 100 is inserted into the interior of an intervertebral disc. Once the tissue is cleaved, stirred, disrupted, and/or severed, the tissue can be removed from the body of the patient.

In some embodiments, the lumen 180 of the elongate member 150 is configured to receive tissue that has been cleaved by the tissue disruptor 167. For example, once the tissue disrupter 167 cleaves the tissue, the tissue can be deposited into the lumen 180. This can occur, for example, by suction applied to a proximal end of the lumen 180. The suction can pull the tissue into the lumen 180. In other embodiments, the lumen 180 can have an opening positioned adjacent the tissue disrupter 167 and the tissue can be deposited into the lumen 180 once the tissue disruptor 167 cleaves the tissue.

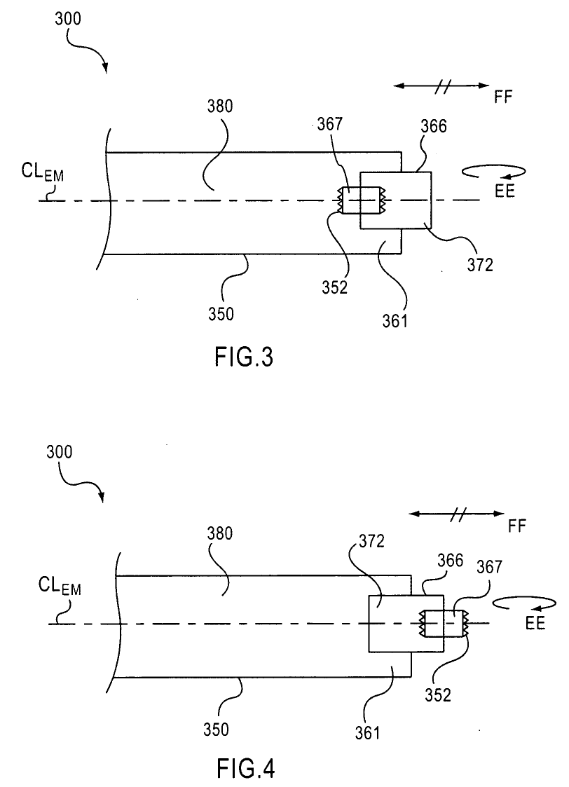

FIG. 2 is a schematic illustration of a medical tool 200, according to an embodiment. Medical tool 200 includes an elongate member 250, a first tissue disruptor 267, and a second tissue disrupter 268. The elongate member 250 has a distal end portion 261 and defines a lumen 280. The distal end portion 261 is configured to be inserted into a body of a patient, as further described herein. The lumen 280 defined by the elongate member 250 defines a center line CL_{EM}. In some embodiments the lumen 280 can be configured to receive body tissue, as further described herein.

The first tissue disruptor 267 of the medical tool 200 is coupled to the distal end portion 261 of the elongate member 250 such that at least a portion of the first tissue disruptor 267 is disposed within the lumen 280. The first tissue disruptor 267 is configured to rotate with respect to the elongate member 250 in the direction shown by arrow CC in FIG. 2. In this manner, the first tissue disruptor 267 can disrupt tissue, as described in more detail herein.

The first tissue disruptor 267 defines a center line CL_{TD1}. As shown in FIG. 2, the center line CL_{TD1} of the first tissue disruptor 267 is offset from the center line CL_{EM} of the lumen 280 of the elongate member 250. The center line CL_{TD1} of the first tissue disruptor 267 is substantially parallel to the center line CL_{EM} of the lumen 280 of the elongate member 250. In alternate embodiments, the center line CL_{TD1} of the first tissue disruptor can be collinear with the center line CL_{EM} of the lumen of the elongate member. In other alternate embodiments, the first tissue disruptor can be positioned such that the center line CL_{TD1} of the first tissue disruptor intersects the center line CL_{EM} of the lumen of the elongate member. In yet other alternate embodiments, the first tissue disruptor can be movable between a first position where the center line CL_{TD1} of the first tissue disruptor is parallel to the center line CL_{EM} of the lumen of the elongate member and a second position where the center line CL_{TD1} of the first tissue disruptor intersects the center line CL_{EM} of the lumen of the elongate member.

The first tissue disruptor 267 is substantially rigid. Said another way, the first tissue disruptor 267 does not substantially deform when rotated within a body of a patient. In alternate embodiments, the first tissue disruptor can be configured to flex and/or bend. Further, while not shown in FIG. 2, the first tissue disruptor 267 can have a sharp cutting surface, for example, a sharp worm gear, a helical flute, and/or claws. Such a sharp cutting surface can aid the first tissue disruptor 267 in disrupting body tissue when the first tissue disruptor 267 of the medical tool 200 is inserted into a body of a patient, as described in further detail below.

Similar to the first tissue disruptor 267 of the medical tool 200, the second tissue disruptor 268 of the medical tool 200 is coupled to the distal end portion 261 of the elongate member 250 such that at least a portion of the second tissue disruptor 268 is disposed within the lumen 280. The second tissue disruptor 268 is configured to rotate with respect to the elongate member 250 in the direction shown by arrow DD in FIG. 2. In this manner, the second tissue disruptor 268 can disrupt tissue, as described in more detail herein.

The second tissue disruptor 268 defines a center line CL_{TD2}. As shown in FIG. 2, the center line CL_{TD2} of the second tissue disruptor 268 is offset from the center line CL_{EM} of the lumen 280 of the elongate member 250. The center line CL_{TD2} of the second tissue disruptor 268 is substantially parallel to the center line CL_{EM} of the lumen 280 of the elongate member 250. In alternate embodiments, the center line CL_{TD2} of the second tissue disruptor can be collinear with the center line CL_{EM} of the lumen of the elongate member. In other alternate embodiments, the second tissue disruptor can be positioned such that the center line CL_{TD2} of the second tissue disruptor intersects the center line CL_{EM} of the lumen of the elongate member. In yet other alternate embodiments, the second tissue disruptor can be movable between a first position where the center line CL_{TD2} of the second tissue disruptor is parallel to the center line CL_{EM} of the lumen of the elongate member and a second position where the center line CL_{TD2} of the second tissue disruptor intersects the center line CL_{EM} of the lumen of the elongate member.

The second tissue disruptor 268 is substantially rigid. Said another way, the second tissue disruptor 268 does not substantially deform when rotated within a body of a patient. In alternate embodiments, the second tissue disruptor can be configured to flex and/or bend. Further, while not shown in FIG. 2, the second tissue disruptor 268 can have a sharp cutting surface, for example, a sharp worm gear, a helical flute, and/or claws. Such a sharp cutting surface can aid the second tissue disruptor 268 in disrupting tissue when the second tissue disruptor 268 of the medical tool 200 is inserted into a body of a patient, as described in further detail below.

In some embodiments, the first tissue disruptor 267 can have a gear and/or helical flute that engages a gear and/or helical flute of the second tissue disruptor 268. In this manner, movement of the first tissue disruptor 267 in a direction defined by the arrow CC in FIG. 2 can cause the second tissue disruptor 268 to move in a direction defined by the arrow DD in FIG. 2, and vice versa. Thus, only one of the first tissue disruptor 267 and the second tissue disruptor 268 needs to be moved to cause both the first tissue disruptor 267 and the second tissue disruptor 268 to move.

In use, the medical tool 200 is inserted into a body of a patient. For example, a medical practitioner can insert the medical tool 200 percutaneously through a cannula into a body of a patient. Similar to the methods described above in relation to medical tool 100, a medical practitioner can gain access to the interior of an intervertebral disc of a patient and insert the medical tool 200 such that the first tissue disruptor 267 and the second tissue disruptor 268 are disposed within the interior of the intervertebral disc of the patient.

Once the first tissue disruptor 267 and the second tissue disruptor 268 of the medical tool 200 are positioned within the body of the patient, the first tissue disruptor 267 is rotated with respect to the elongate member 250 in the direction shown by the arrow CC in FIG. 2 and the second tissue disruptor 268 is rotated with respect to the elongate member 250 in the direction shown by the arrow DD in FIG. 2. By rotating the first tissue disruptor 267 in the direction shown by the arrow CC in FIG. 2 and the second tissue disruptor 268 in the direction shown by the arrow DD in FIG. 2, the body tissue adjacent the first tissue disruptor 267 and/or the second tissue disruptor 268 is cleaved, stirred, disrupted, and/or severed. For example, the first tissue disruptor 267 and/or the second tissue disruptor 268 can cleave, stir, disrupt, and/or sever at least a portion of the nucleus of an intervertebral disc when the medical tool 200 is inserted into the interior of an intervertebral disc. Once the body tissue is cleaved, stirred, disrupted, and/or severed, the body tissue can be removed from the body of the patient.

In some embodiments, the lumen 280 of the elongate member 250 is configured to receive the body tissue that is cleaved by the first tissue disruptor 267 and/or the second tissue disruptor 268. For example, once the first tissue disruptor 267 and/or the second tissue disruptor 268 cleaves the body tissue, it can be deposited into the lumen 280. This can occur, for example, by suction applied to a proximal end of the lumen 280. The suction can pull the tissue into the lumen 280. In other embodiments, the lumen 280 can have an opening positioned adjacent the first tissue disruptor 267 and/or the second tissue disruptor 268, and the tissue can be deposited into the lumen 280 once the first tissue disruptor 267 and/or the second tissue disruptor 268 cleaves the tissue.

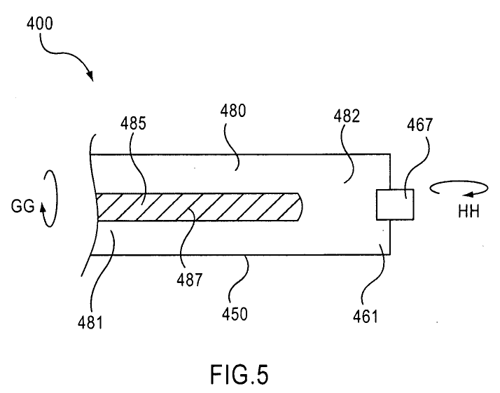

FIGS. 3 and 4 are schematic illustrations of a medical tool 300 in a first configuration and a second configuration, respectively, according to an embodiment. Medical tool 300 includes an elongate member 350 and a tissue disruptor 366. The elongate member 350 has a distal end portion 361 and defines a lumen 380. The distal end portion 361 is configured to be inserted into a body of a patient, as further described herein. The lumen 380 defined by the elongate member 350 defines a center line CL_{EM}. In some embodiments the lumen 380 can be configured to receive body tissue, as further described herein.

The tissue disrupter 366 of the medical tool 300 includes a carriage 372 and a rotatable member 367, and is coupled to the distal end portion 361 of the elongate member 350 such that movement of the tissue disrupter 366 relative to the elongate member 350 in the direction shown by arrow FF in FIGS. 3 and 4 is limited and/or prohibited. The carriage 372 is rotatably coupled to the distal end portion 361 of the elongate member 350, and is configured to rotate relative to the elongate member 350 in a direction shown by the arrow EE in FIGS. 3 and 4. When the carriage 372 rotates in the direction shown by the arrow EE in FIGS. 3 and 4, the carriage 372 is configured to move between a first position (FIG. 3) and a second position (FIG. 4), as further described herein.

The rotatable member 367 of the tissue disrupter 366 is coupled to the carriage 372 and has a cutting surface 352. The rotatable member 367 is configured to rotate relative to the carriage 372 in a direction shown by the arrow EE in FIGS. 3 and 4. In some embodiments, the cutting surface 352 can have a sharp edge. For example, the cutting surface 352 can include a sharp worm gear, a helical flute, and/or claws. The cutting surface 352 can be configured to disrupt tissue when the rotatable member 367 of the tissue disrupter 366 is inserted into a body of a patient, as described in further detail below. In some embodiments, the rotatable member 367 of the tissue disrupter 366 can be substantially rigid. In other embodiments, the rotatable member can be configured to flex and/or bend.

As shown in FIGS. 3 and 4, the carriage 372 of the tissue disrupter 366 is movable between a first position (FIG. 3) and a second position (FIG. 4). When the carriage 372 of the tissue disrupter 366 is in the first position, the cutting surface 352 of the rotatable member 367 is disposed within the lumen 380 defined by the elongate member 350. Said another way, when the carriage 372 of the tissue disrupter 366 is in the first position, the cutting surface 352 of the rotatable member 367 is not exposed to the area surrounding the distal end portion 361 of the elongate member 350.

To move the carriage 372 of the tissue disrupter 366 from the first position to the second position, the carriage 372 is rotated with respect to the elongate member 350 in the direction shown by the arrow EE in FIGS. 3 and 4. When the carriage 372 of the tissue disrupter 366 is rotated in the direction shown by the arrow EE in FIGS. 3 and 4 and into the second position, at least a portion of the cutting surface 352 of the rotatable member 367 is disposed outside of the lumen 380 defined by the elongate member 350. Said another way, when the carriage 372 of the tissue disrupter 366 is in the second position, the cutting surface 352 of the rotatable member 367 is exposed to the area surrounding the distal end portion 361 of the elongate member 350.

In use, the medical tool 300 is inserted into a body of a patient with the carriage 372 of the tissue disrupter 366 in the first position. More specifically, the tissue disrupter 366 is inserted into a body of a patient when the cutting surface 352 of the tissue disrupter 366 is not exposed to the area surrounding the distal end portion 361 of the elongate member 350. For example, a medical practitioner can insert the medical tool 300 percutaneously through a cannula into a body of a patient. Similar to the methods described above in relation to medical tool 100, a medical practitioner can gain access to the interior of an intervertebral disc of a patient and insert the medical tool 300 such that the tissue disrupter 366 is disposed within the interior of the intervertebral disc of the patient.

By inserting the medical tool 300 into the body of the patient when the carriage 372 of the tissue disrupter 366 is in the first position, minimal harm is done to the body of the patient. Because the cutting surface 352 of the tissue disrupter 366 is not exposed to the area surrounding the distal end portion 361 of the elongate member 350 when the carriage 372 of the tissue disrupter 366 is in the first position, the cutting surface 352 does not contact the tissue surrounding the distal end portion 361 of the elongate member 350 during insertion. For example, the medical tool 300 can be safely inserted into the interior of an intervertebral disc without the cutting surface 352 contacting the annulus of the disc. Thus, the tissue disrupter 366 can be inserted into the intervertebral disc of the patient without the cutting surface 352 damaging the annulus.

Once the tissue disrupter 366 of the medical tool 300 is positioned within the body of the patient, the carriage 372 of the tissue disrupter 366 is moved from the first position to the second position as described above. Moving the carriage 372 of the tissue disrupter 366 exposes the cutting surface 352 of the rotatable member 367 to the area surrounding the distal end portion 361 of the elongate member 350. For example, when medical tool 300 is inserted into the interior of an intervertebral disc, the carriage 372 of the tissue disrupter 366 can be moved to the second position to expose the cutting surface 352 of the rotatable member 367 to the nucleus of the intervertebral disc.

Once the carriage 372 of the tissue disrupter 366 is in the second position, the rotatable member 367 can be rotated with respect to the carriage 372 in the direction shown by the arrow EE in FIG. 4. By rotating the rotatable member 367 in the direction shown by the arrow EE in FIG. 4, the cutting surface 352 of the rotatable member 367 contacts and cleaves, stirs, disrupts, and/or severs the body tissue adjacent the rotatable member 367. For example, the cutting surface 352 of the rotatable member 367 can cleave, stir, disrupt, and/or sever at least a portion of the nucleus of an intervertebral disc when the tissue disrupter 366 is inserted into the interior of an intervertebral disc. Once the body tissue is cleaved, stirred, disrupted, and/or severed, the body tissue can be removed from the body of the patient.

In some embodiments, the lumen 380 of the elongate member 350 is configured to receive the tissue that is severed by the cutting surface 352 of the rotatable member 367. For example, once the cutting surface 352 of the rotatable member 367 severs the body tissue, it can be deposited into the lumen 380. This can occur, for example, by suction applied to a proximal end of the lumen 380. The suction can pull the tissue into the lumen 380. In other embodiments, the lumen can have an opening positioned adjacent the rotatable member and the body tissue can be deposited into the lumen once the rotatable member severs the body tissue.

Once the cutting surface 352 of the rotatable member 367 has severed the body tissue, the medical tool 300 can be removed from the body of the patient. The medical tool 300 is removed from the body of the patient by first rotating the carriage 372 of the tissue disrupter 366 in the direction shown by the arrow EE in FIGS. 3 and 4. This moves the carriage 372 of the tissue disrupter 366 from the second position to the first position. As discussed above, when the carriage 372 of the tissue disrupter 366 is in the first position, the cutting surface 352 of the rotatable member 367 is disposed within the lumen 380 defined by the elongate member 350 and does not contact the area surrounding the distal end portion 361 of the elongate member 350. Once the carriage 372 of the tissue disrupter 366 is in the first position, the medical tool 300 can be safely removed from the body of the patient.

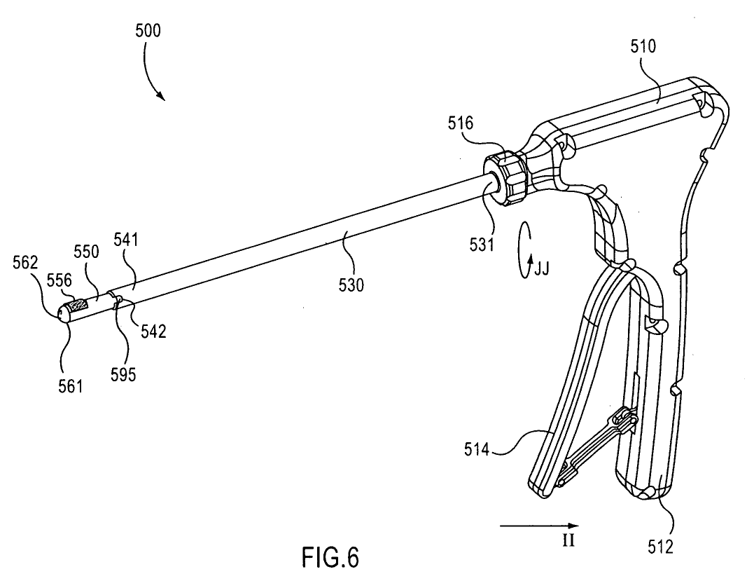

FIG. 5 is a schematic illustration of a medical tool 400, according to another embodiment. Medical tool 400 includes an elongate member 450, a tissue disrupter 467, and a threaded member 485. The elongate member 450 has a distal end portion 461 and defines a lumen 480. The distal end portion 461 is configured to be inserted into a body of a patient, as further described herein. The lumen 480 defined by the elongate member 450 includes a distal portion 482 and a proximal portion 481 and is configured to receive body tissue, as further described herein.

The tissue disrupter 467 of the medical tool 400 is coupled to the distal end portion 461 of the elongate member 450 and is configured to rotate with respect to the elongate member 450 in the direction shown by arrow HH in FIG. 5. The tissue disrupter 467 can be similar to the rotating members described in U.S. application Ser. No. 11/448,976 filed on Jun. 8, 2006 and entitled “Dual Cutting Element Tool for Debulking Bone,” which is incorporated herein by reference in its entirety. In this manner, the tissue disrupter 467 is configured to convey a body tissue from outside the elongate member 450 into the distal portion 482 of the lumen 480 defined by the elongate member 450, as described in more detail herein. In some embodiments, the tissue disrupter 467 can disrupt body tissue, prior to conveying the body tissue from outside the elongate member 450 into the distal portion 482 of the lumen 480.

In some embodiments, the tissue disrupter 467 is substantially rigid. Said another way, the first tissue disrupter 467 does not substantially deform when rotated within a body of a patient. In alternate embodiments, the tissue disrupter can be configured to flex and/or bend. Further, while not shown in FIG. 5, in some embodiments, the tissue disrupter 467 can have a sharp cutting surface for example, a sharp worm gear, a helical flute, and/or claws. Such a sharp cutting surface can aid the tissue disrupter 467 in disrupting tissue when the tissue disrupter 467 of the medical tool 400 is inserted into a body of a patient.

The threaded member 485 includes one or more threads 487 and is disposed within the lumen 480 defined by the elongate member 450. The threaded member 485 is disposed within the lumen 480 such that a portion of the threaded member 485 is disposed within the proximal portion 481 of the lumen 480 and a portion of the threaded member 485 is disposed within the distal portion 482 of the lumen 480. The threaded member 485 is configured to rotate with respect to the elongate member 450 in the direction shown by the arrow GG in FIG. 5. When the threaded member 485 rotates in the direction shown by the arrow GG in FIG. 5, the threads 487 of the threaded member 485 are configured to convey a body tissue from the distal portion 482 of the lumen 480 to the proximal portion 481 of the lumen 480, as further described herein. In some embodiments, for example, the threaded member 485 can be an Archimedes screw.

In some embodiments, the threaded member can be connected to the tissue disruptor 467 by a drive shaft and/or a gear system. In this manner, when the threaded member is rotated in the direction shown by the arrow GG in FIG. 5, the tissue disruptor is also rotated, and vise versa. Thus, only one motor is needed to rotate both the threaded member and the tissue disruptor.

In use, the medical tool 400 is inserted into a body of a patient. For example, a medical practitioner can insert the medical tool 400 percutaneously through a cannula into a body of a patient. Similar to the methods described above in relation to medical tool 100, a medical practitioner can gain access to the interior of an intervertebral disc of a patient and insert the medical tool 400 such that the tissue disruptor 467 is disposed within the interior of the intervertebral disc of the patient.

Once the tissue disruptor 467 of the medical tool 400 is positioned within the body of the patient, the tissue disrupter 467 is rotated with respect to the elongate member 450 in the direction shown by the arrow HH in FIG. 5. By rotating the tissue disruptor 467 in the direction shown by the arrow HH in FIG. 5, the body tissue adjacent the tissue disruptor 467 is conveyed from the body and into the distal portion 482 of the lumen 480. Said another way, when the tissue disrupter 467 is rotated, the body tissue is collected in the distal portion 482 of the lumen 480.

In some embodiments, the tissue disrupter 467 can cleave, stir, disrupt, and/or sever the body tissue before the tissue disrupter 467 conveys the body tissue into the distal portion 482 of the lumen 480. Once the body tissue is cleaved, stirred and/or severed, the body tissue can be collected in the distal portion 482 of the lumen 480.

Once the tissue is collected in the distal portion 482 of the lumen 480, the threaded member 485 is rotated in the direction shown by the arrow GG in FIG. 5. The threads 487 of the threaded member 485 engage the tissue collected in the distal portion 482 of the lumen 480 and convey the tissue from the distal portion 482 of the lumen 480 to the proximal portion 481 of the lumen 480. Once the tissue is in the proximal portion 481 of the lumen 480, the tissue can be removed from the lumen 480.

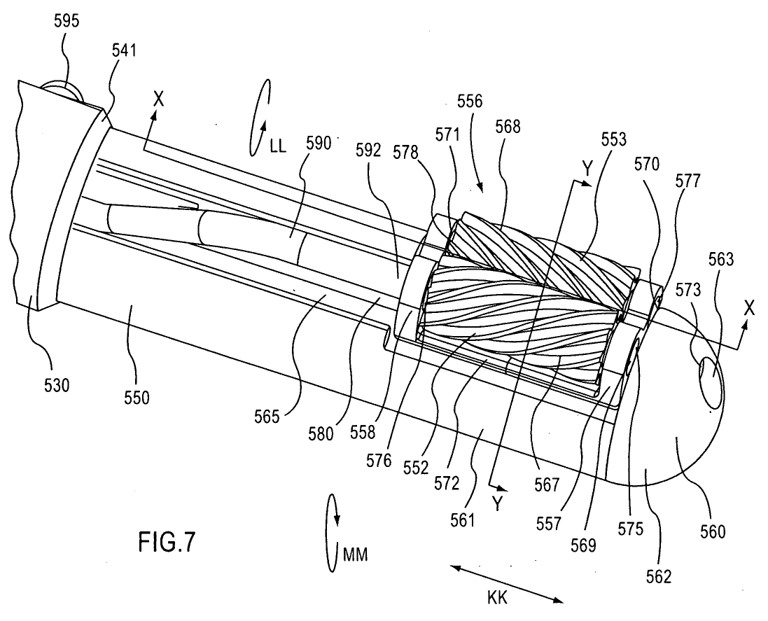

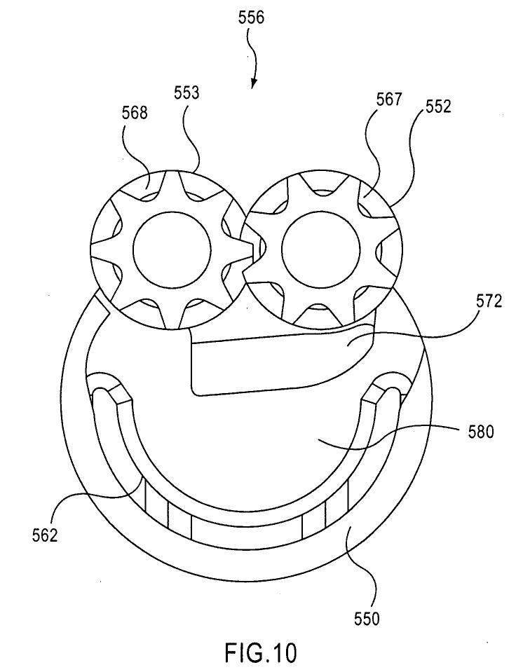

FIGS. 6-10 show a medical tool 500, according to another embodiment. Medical tool 500 includes a housing 510, an outer elongate member 530, an inner elongate member 550, a tissue disrupter 556, a threaded member 585, a flexible shaft 590 and a distal cap 562. The inner elongate member 550 is partially disposed within a lumen 545 defined by the outer elongate member 530. The inner elongate member 550 includes a proximal end portion (not shown), a distal end portion 561, and defines a first lumen 580, a second lumen 564 and a side aperture 565. The proximal end portion is configured to be fixedly coupled to the housing 510, as further described herein. The second lumen 564 of the elongate member 550 receives and rotatably retains the second protrusion 574 of the carriage 572, as further described herein. The flexible shaft 590 is disposed within the side aperture 565, as further described herein. The first lumen 580 of the inner elongate member 550 is configured to receive and collect body tissue when the tissue disrupter disrupts body tissue, as further described herein.

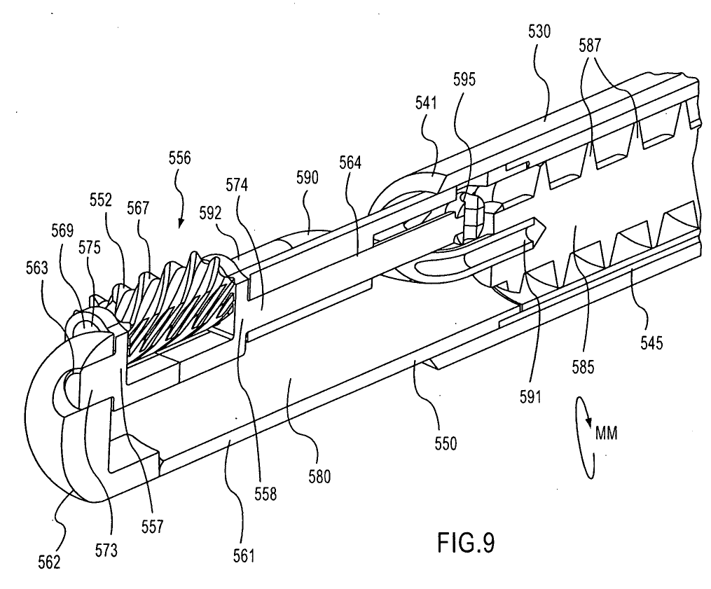

The threaded member 585 is rotatably disposed within the first lumen 580 of the inner elongate member 550 and includes threads 587, which are configured to convey tissue disposed within the inner elongate member 550 from the distal end portion 561 of the inner elongate member 550 to the proximal end portion of the inner elongate member 550 when the threaded member 585 rotates relative to the inner elongate member 550 in a direction shown by the arrow MM in FIG. 9, as further described herein. In some embodiments, for example, the threaded member can be an Archimedes screw.

The flexible shaft 590 of the medical tool 500 includes a proximal end portion 591 and a distal end portion 592. The proximal end portion 591 of the flexible shaft 590 is coupled to the threaded member 585. As such, when the threaded member 585 rotates in the direction shown by the arrow MM in FIG. 9, the flexible shaft 590 rotates in the direction shown by the arrow MM in FIG. 9.

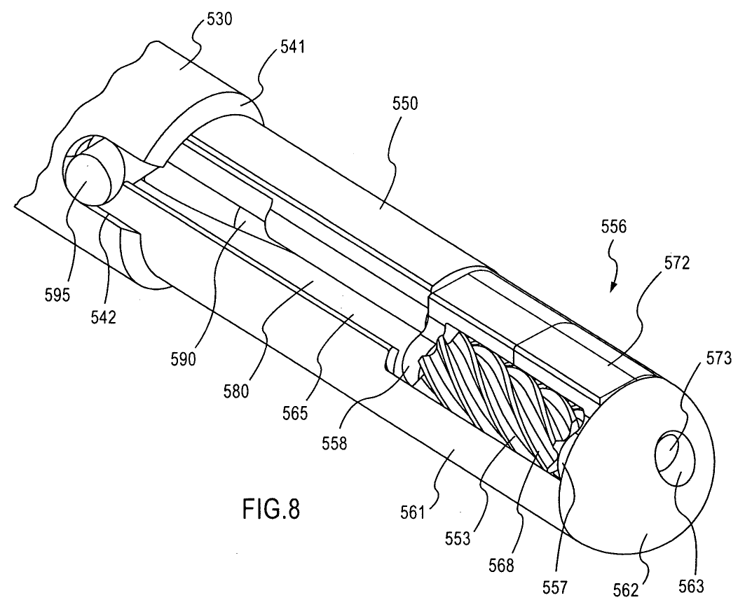

The flexible shaft 590 is disposed within the side aperture 565 (best shown in FIG. 7) defined by the inner elongate member 550, when the carriage 572 is in its first configuration, as described in further detail herein. The distal end portion 592 of the flexible shaft 590 is coupled to a first rotatable member 567 of the tissue disrupter 556 such that when the flexible shaft 590 rotates in the direction shown by the arrow MM in FIG. 9, the first rotatable member 567 similarly rotates in the direction shown by the arrow MM in FIG. 9. Thus, rotating the threaded member 585 in the direction shown by the arrow MM in FIG. 9, causes the first rotatable member 567 to rotate in the direction shown by the arrow MM in FIG. 9. As described in further detail herein, this causes a second rotatable member 568 to rotate in the direction shown by the arrow LL in FIG. 7.

The distal cap 562 of the medical tool 500 is coupled to the distal end portion 561 of the inner elongate member 550. The distal cap 562 includes an insertion surface 560 and defines a lumen 563. The insertion surface 560 of the distal cap 562 is configured to be inserted first when the medical tool 500 is inserted into the body of a patient. As such, the insertion surface 560 of the distal cap 562 is rounded (or any atraumatic shape) such that it does not harm tissue when the medical tool 500 is inserted into the body of a patient. In other embodiments, the insertion surface can be configured to pierce a body tissue to facilitate insertion. The lumen 563 defined by the distal cap 562 receives and rotatably retains the first protrusion 573 of the carriage 572, as further described herein.

The tissue disrupter 556 of the medical tool 500 includes a carriage 572, a first rotatable member 567 and a second rotatable member 568. The tissue disrupter 556 is coupled to the distal end portion 561 of the inner elongate member 550 such that movement of the tissue disrupter 556 relative to the inner elongate member 550 in the direction shown by the arrow KK in FIG. 7 is limited or prohibited. Said another way, the tissue disrupter 556 does not substantially move relative to the inner elongate member 550 in a longitudinal direction.

The carriage 572 includes a distal end portion 557 and a proximal end portion 558 and is configured to move between a first configuration and a second configuration. The first rotatable member 567 and the second rotatable member 568 are configured to be disposed between the distal end portion 557 of the carriage 572 and the proximal end portion 558 of the carriage 572. The distal end portion 557 of the carriage 572 includes a first protrusion 573, a first aperture 575, and a third aperture 577. The proximal end portion 558 includes a second protrusion 574, a second aperture 576, and a fourth aperture 578.

The carriage 572 is rotatably coupled to the distal end portion 561 of the inner elongate member 550 by the first protrusion 573 and the second protrusion 574 such that the carriage can rotate between a first configuration (FIG. 7) and a second configuration (FIG. 8), as described in further detail herein. More specifically, the first protrusion 573 of the carriage 572 is disposed and/or rotatably retained within the lumen 563 defined by the distal cap 562, and the second protrusion 574 of the carriage 572 is disposed and/or rotatably retained within the second lumen 564 defined by the inner elongate member 550. The first protrusion 573 and the second protrusion 574 are configured to rotate within the lumen 563 defined by the distal cap 562 and the second lumen 564 defined by the inner elongate member 550, respectively. Such rotation of the first protrusion 573 and the second protrusion 574 causes the carriage 572 to move between the first configuration and the second configuration.

The second protrusion 574 is attached to a pivot rod 595 that is disposed through a side wall of the inner elongate member 550. The pivot rod 595 is configured to be disposed within a notch 542 of the outer elongate member 530 (best seen in FIG. 8), as further described herein. When the pivot rod 595 moves from a first position (FIG. 7) to a second position (FIG. 8), the first protrusion 573 of the carriage 572 and the second protrusion 574 of the carriage 572 rotate in the direction shown by the arrow LL in FIG. 7. This causes the carriage 572 to move from the first configuration to the second configuration. Similarly, when the pivot rod 595 moves from its second position (FIG. 8) to its first position (FIG. 7), the first protrusion 573 of the carriage 572 and the second protrusion 574 of the carriage 572 rotate in the direction shown by the arrow MM in FIG. 7. This causes the carriage 572 to move from the second configuration to the first configuration. Said another way, the pivot rod 595 controls whether the carriage 572 is in its first configuration or its second configuration.

The carriage 572 rotatably retains the first rotatable member 567. More specifically, the first rotatable member 567 is disposed between the distal end portion 557 of the carriage 572 and the proximal end portion 558 of the carriage 572. The first aperture 575 of the carriage 572 receives a protrusion 569 of the first rotatable member 567, and the second aperture 576 of the carriage 572 receives a distal end portion 592 of the flexible shaft 590 that is coupled to the first rotatable member 567, as further described herein. The protrusion 569 of the first rotatable member 567 and the flexible shaft 590 are configured to rotate within the first aperture 575 of the carriage 572 and the second aperture 576 of the carriage 572, respectively. In this manner the first rotatable member 567 is rotatably retained within the carriage 572.

Similarly, the carriage 572 rotatably retains the second rotatable member 568. More specifically, the second rotatable member 568 is disposed between the distal end portion 557 of the carriage 572 and the proximal end portion 558 of the carriage 572. The third aperture 577 of the carriage 572 receives a first protrusion 570 of the second rotatable member 568, and the fourth aperture 578 of the carriage 572 receives a second protrusion 571 of the second rotatable member 568. The first protrusion 570 of the second rotatable member 568 and the second protrusion 571 of the second rotatable member 568 are configured to rotate within the third aperture 577 of the carriage 572 and the fourth aperture 578 of the carriage 572, respectively. In this manner the second rotatable member 568 is rotatably retained within the carriage 572.

When the carriage 572 is in its first configuration (FIG. 7), the first rotatable member 567 and the second rotatable member 568 are only partially disposed within the first lumen 580 defined by the inner elongate member 550. The first rotatable member 567 and the second rotatable member 568 are exposed to the area surrounding the distal end portion 561 of the inner elongate member 550, and thus body tissue when the medical tool 500 is inserted into a body of a patient. When the carriage 572 is in its second configuration (FIG. 8), the first rotatable member 567 and the second rotatable member 568 are entirely disposed within the first lumen 580 defined by the inner elongate member 550. Said another way, when the carriage is in its second configuration, the first rotatable member 567 and the second rotatable member 568 are not exposed to the area surrounding the distal end portion 561 of the inner elongate member 550.

In some embodiments, the first rotatable member and/or the second rotatable member can be entirely disposed outside the lumen defined by the elongate member when the carriage is in its first configuration as long as the first rotatable member and the second rotatable member can disrupt tissue and deposit the disrupted tissue into the first lumen defined by the elongate member, as further described in detail herein. Similarly, in some embodiments, the first rotatable member and/or the second rotatable member can be only partially disposed within the lumen defined by the elongate member when the carriage is in its second configuration as long as the first rotatable member and the second rotatable member do not significantly disrupt tissue during insertion, as further described in detail herein.

The first rotatable member 567 of the tissue disrupter 556 is substantially cylindrical in shape and includes a cutting surface 552 and a protrusion 569. The first rotatable member 567 is substantially rigid. Said another way, the first rotatable member 567 does not substantially deform when rotated within a body of a patient. As described above, the first rotatable member 567 is disposed between the distal end portion 557 of the carriage 572 and the proximal end portion 558 of the carriage 572.

The cutting surface 552 of the first rotatable member 567 includes a helical flute configured to engage a helical flute of a cutting surface 553 of the second rotatable member 568, as further described herein. The helical flute of the cutting surface 552 is sharp and configured to cleave, stir, disrupt, and/or sever body tissue when the first rotatable member 567 of the first tissue disrupter 556 is inserted into a body of a patient, as described in further detail below.

The first rotatable member 567 is configured to rotate with respect to the carriage 572 in a direction shown by the arrow MM in FIG. 7. When the first rotatable member 567 rotates within a body of a patient, the cutting surface 552 of the first rotatable member 567 is configured to cleave, stir, disrupt, and/or sever body tissue disposed within the body of the patient.

Similar to the first rotatable member 567, the second rotatable member 568 of the tissue disrupter 556 is substantially cylindrical in shape and includes a cutting surface 553, a first protrusion 570 and a second protrusion 571. The second rotatable member 568 is substantially rigid. Said another way, the second rotatable member 568 does not substantially deform when rotated within a body of a patient. As described above, the second rotatable member 568 is disposed between the distal end portion 557 of the carriage 572 and the proximal end portion 558 of the carriage 572.

The cutting surface 553 of the second rotatable member 568 includes a helical flute configured to engage the helical flute on the cutting surface 552 of the first rotatable member 567, as further described herein. The helical flute of the cutting surface 553 is sharp and configured to cleave, stir, disrupt, and/or sever body tissue.

The second rotatable member 568 is configured to rotate with respect to the carriage 572 in the direction shown by the arrow LL in FIG. 7. When the second rotatable member 568 rotates within a body of a patient, the cutting surface 553 of the second rotatable member 568 is configured to cleave, stir, disrupt, and/or sever body tissue disposed within the body of the patient.

As described above, the helical flute of the cutting surface 552 of the first rotatable member 567 is configured to engage the helical flute of the cutting surface 553 of the second rotatable member 568. As such, the first rotatable member 567 and the second rotatable member 568 act as opposing gears. Said another way, when the first rotatable member 567 rotates relative to the carriage 572 in the direction shown by the arrow MM in FIG. 7, the second rotatable member 568 rotates in the direction shown by the arrow LL in FIG. 7. Said yet another way, rotating the first rotatable member 567 relative to the carriage 572 in a first direction (e.g., counter-clockwise), causes the second rotatable member 568 to rotate relative to the carriage 572 in a second direction, opposite the first direction (e.g., clockwise). As described above, rotation of the first rotatable member 567 and the second rotatable member 568 cleaves, stirs, disrupts, and/or severs body tissue adjacent the distal end portion 561 of the inner elongate member 550. In alternate embodiments, the first rotatable member and the second rotatable member can be configured to rotate in the direction opposite the direction shown by the arrow MM in FIG. 7 and the direction opposite the direction shown by the arrow LL in FIG. 7, respectively.

As the first rotatable member 567 and the second rotatable member 568 rotate, tissue passes between the first rotatable member 567 and the second rotatable member 568. As the tissue passes between the first rotatable member 567 and the second rotatable member 568, the tissue is further cleaved, stirred, disrupted, and/or severed. Once the tissue passes between the first rotatable member 567 and the second rotatable member 568, the tissue is deposited into the first lumen 580 defined by the inner elongate member 550, as described in further detail herein.

The outer elongate member 530 of the medical device 500 includes a proximal end portion 531, a distal end portion 541 and defines a lumen 545. As described above, a portion of the inner elongate member 550, including the proximal end portion of the inner elongate member 550, is disposed within the outer elongate member 530. The distal end portion 561 of the inner elongate member 550 is not disposed within the outer elongate member 530.

The proximal end portion 531 of the outer elongate member 530 is coupled to a carriage actuator 516 of the housing 510. The carriage actuator 516 of the housing is configured to rotate the outer elongate member 530 with respect to the inner elongate member 550, between a first position and a second position, as further described herein.

The distal end portion 531 of the outer elongate member 530 includes a notch 542 configured to receive the pivot rod 595. When the outer elongate member 530 moves from its first position to its second position, the notch 542 causes the pivot rod 595 to move from its first position to its second position causing the carriage 572 to move from the first configuration to the second configuration, as described above.

The housing 510 includes a handle 512, an actuation lever 514, a conversion mechanism (not shown) and a carriage actuator 516. The housing 510 is similar to the housing described in U.S. patent application Ser. No. 12/109,565 filed Apr. 25, 2008 and entitled “Medical Device With One-Way Rotary Drive Mechanism,” which is incorporated herein by reference in its entirety. As such, the housing 510 is not described in detail herein.

As shown in FIG. 6, the actuation lever 514 of the housing 510 is coupled to the handle 512 of the housing 510. The actuation lever 514 of the housing 510 is also coupled to the conversion mechanism, which is disposed within the housing 510.

The actuation lever 514 has a first position where a distal end of the actuation lever 514 is spaced apart from the handle 512 by a first distance, and a second position where the distal end of the actuation lever 514 is spaced apart from the handle 512 by a second distance, less than the first. The actuation lever 514 is biased in the first position. By moving the actuation lever 514 relative to the handle 512 in a direction shown by the arrow II in FIG. 6, a user can move the actuation lever 514 from the first position to the second position. When the actuation lever 514 is moved from its first position to its second position, the conversion mechanism rotates the threaded member 585 in the direction shown by the arrow MM in FIG. 9.

The conversion mechanism of the housing 510 converts translational motion generated via actuation lever 514 (e.g., by the squeezing of the actuation lever 514 toward the handle 28) into rotational motion of the threaded member 585. The conversion mechanism allows a user of medical tool 500 to generate rotational torque and motion to tissue disrupter 556 without having to repeatedly twist his/her arm, as would be required by conventional medical tools.

In some embodiments, the conversion mechanism can include a threaded drive element (not shown) configured to engage a threaded portion (not shown) of a component (not shown) coupled to the threaded member 585. In some embodiments, the threaded portion can be, for example, a lead screw. The threaded drive element can include a lead nut (not shown in) and a face gear (not shown). In some embodiments, the drive element can alternatively include other components, such as for example, a drive nut, a gear, a pulley system, and/or a split nut. The conversion mechanism can further include a return spring, a bronze bearing, and a pair of thrust bearings (not shown). The medical tool 500 can also include a rotation-limiting mechanism for allowing rotation of the threaded member 585 in only a single direction. The rotation-limiting mechanism can be, for example, a roller or rotary clutch (not shown), or other ratcheting mechanism.

The carriage actuator 516 of the housing 510 is coupled to the outer elongate member 530. The carriage actuator 516 is configured to rotate with respect to the housing 510 in a first direction as shown by the arrow JJ in FIG. 6 and a second direction, opposite the first. When the carriage actuator 516 rotates in the first direction, the outer elongate member 530 rotates in the first direction causing the pivot rod 595 to rotate in the first direction. This causes the carriage 572 of the tissue disrupter 556 to move from the first configuration to the second configuration, as described above. Similarly, when the carriage actuator 516 rotates in the second direction, the outer elongate member 530 rotates in the second direction causing the carriage 572 of the tissue disrupter 556 to move from the second configuration to the first configuration.

In some embodiments, the housing 510 can include a collection vessel. The collection vessel can be in fluid communication with the first lumen 580 defined by the inner elongate member 550. In this manner, the collection vessel collects tissue as the tissue is disrupted and moved in a proximal direction by the threaded member 585, as further described herein. In some embodiments, the collection vessel includes a one-way valve, such as a pressure relief valve, configured to allow for air to escape from within the collection vessel. For example, in some embodiments, as tissue fragments are drawn into the collection vessel, air within the collection vessel may become pressurized. A pressure relief valve can be used to allow for a one-way flow of air to exit the collection vessel as tissue is moved into the collection vessel.

To actuate the tissue disrupter 556, a user moves the lever 514 in a direction shown by the arrow II in FIG. 6 from its first position to its second position. As discussed above, when the actuation lever 514 is moved from its first position to its second position, the conversion mechanism (not shown) converts the translational motion of the actuation lever 514 into rotational motion, which causes the threaded member 585 to rotate in the direction shown by the arrow MM in FIG. 9. Because the proximal end 591 of the flexible shaft 590 is coupled to the threaded member 585, rotation of the threaded member 585 in the direction shown by the arrow MM in FIGS. 7 and 9 causes the flexible shaft 590 to rotate in the direction shown by the arrow MM in FIGS. 7 and 9. As described above, the flexible shaft 590 is coupled to the first rotatable member 567. As such, when the flexible shaft 590 rotates in the direction shown by the arrow MM in FIGS. 7 and 9, the first rotatable member 567 similarly rotates. Because the helical flute of the first rotatable member 567 engages the helical flute of the second rotatable member, the first rotatable member 567 causes the second rotatable member 568 to rotate in the direction shown by the arrow LL in FIG. 7. Thus, moving the actuation lever 514 of the housing 510 from its first position to its second position causes the first rotatable member 567 and the second rotatable member 568 to rotate.

Once the user releases the actuation lever 514, the actuation lever 514 moves from its second position to its first position. The conversion mechanism, however, does not convert this translational motion into rotational motion. Thus, unlike moving the actuation lever 514 of the housing 510 from its first position to its second position, moving the actuation lever 514 of the housing 510 from its second position to its first position does not cause the first rotatable member 567 and the second rotatable member 568 to rotate.

In use, the medical tool 500 is inserted into a body of a patient with the carriage 572 of the tissue disrupter 556 in the second configuration. More specifically, the tissue disrupter 556 is inserted into a body of a patient when the first rotatable member 567 of the tissue disrupter 556 and the second rotatable member 568 of the tissue disrupter 556 are not exposed to the area surrounding distal end portion 561 of the inner elongate member 550. For example, a medical practitioner can insert the medical tool 500 percutaneously through a cannula into a body of a patient. Similar to the methods described above in relation to medical tool 100, a medical practitioner can gain access to the interior of an intervertebral disc of a patient and insert the medical tool 500 such that the tissue disrupter 556 is disposed within the interior of the intervertebral disc of the patient.

By inserting the medical tool 500 into the body of the patient with the carriage 572 of the tissue disrupter 556 in the first configuration, minimal harm is done to the body of the patient. Because the cutting surface 552 of the first rotatable member 567 and the cutting surface 553 of the second rotatable member are not exposed to the area surrounding the distal end portion 561 of the inner elongate member 550 when the carriage 572 of the tissue disrupter 556 is in the first configuration, the cutting surfaces 552, 553 cannot contact the tissue surrounding the elongate member 550 during insertion. For example, the medical tool 500 can be safely inserted into the interior of an intervertebral disc without the cutting surfaces 552, 553 contacting the annulus of the disc. Thus, the tissue disrupter 556 can be inserted into the intervertebral disc of the patient without the cutting surfaces 552, 553 damaging the annulus.

Once the tissue disrupter 556 of the medical tool 500 is positioned within the body of the patient, the carriage 572 of the tissue disrupter 556 is moved from the second configuration (FIG. 8) to the first configuration (FIG. 7). As described above, to move the carriage 572 of the tissue disrupter 556 from the second configuration to the first configuration, the carriage actuator 516 is rotated in a direction opposite the direction shown by the arrow JJ in FIG. 6, causing the outer elongate member 530 to similarly rotate. This causes the notch 542 of the distal end portion 541 of the outer elongate member 530 to contact the pivot rod 595, causing the carriage to rotate in the direction shown by the arrow MM in FIG. 7 and into the first configuration.

Moving the carriage 572 of the tissue disrupter 556 exposes the cutting surface 552 of the first rotatable member 567 and the cutting surface 553 of the second rotatable member 568 to the area surrounding the medical tool 500. For example, when the medical tool 500 is inserted into the interior of an intervertebral disc, the carriage 572 of the tissue disrupter 556 can be moved to the second position to expose the cutting surfaces 552, 553 to the nucleus of the intervertebral disc.

Once the carriage 572 of the tissue disrupter 556 is in the first configuration, the first rotatable member 567 and the second rotatable member 568 can be rotated with respect to the carriage 572 in the directions shown by the arrows MM and LL in FIG. 7, respectively. As discussed above, this is accomplished by moving the actuation lever 514 of the housing 510 from its first position to its second position. To achieve continual motion of the first rotatable member 567 and the second rotatable member 568, the user can repeatedly move the actuation lever 514 between its first position and its second position.

By rotating the first rotatable member 567 in the direction shown by the arrow MM in FIG. 7 and the second rotatable member 568 in the direction shown by the arrow LL in FIG. 7, the cutting surface 552 of the first rotatable member 567 and the cutting surface 553 of the second rotatable member 568 contact and cleave, stir, disrupt, and/or sever the body tissue adjacent the cutting surfaces 552, 553. For example, the cutting surface 552 of the first rotatable member 567 and/or the cutting surface 553 of the second rotatable member 568 can cleave, stir, disrupt, and/or sever at least a portion of the nucleus of an intervertebral disc when the medical tool 500 is inserted into the interior of an intervertebral disc.

Once the body tissue is cleaved, stirred, disrupted, and/or severed, the body tissue can be conveyed between the first rotatable member 567 and the second rotatable member 568 and into the first lumen 580 defined by the inner elongate member 550. As more tissue is deposited into the first lumen 580 defined by the inner elongate member 550, the tissue begins to move in a proximal direction from the distal end portion 561 of the inner elongate member 550.

As the tissue moves in a proximal direction, the tissue contacts the threaded member 585. As described above, when the actuation lever 514 is moved between its first position and its second position, the threaded member 585 rotates in the direction shown by the arrow MM in FIG. 9. Said another way, the threaded member 585 simultaneously rotates with the first rotatable member 567 and the second rotatable member 568. The threads 587 of the threaded member 585 contact the tissue and are configured to move the tissue away from the distal end portion 561 of the inner elongate member 550, when the threaded member rotates in the direction shown by the arrow MM in FIG. 9.

Once the tissue has been removed from the body of the patient, the medical tool 500 can be removed from the body of the patient. To remove the medical tool 500, the carriage 572 is moved from its first configuration to its second configuration. This is done by rotating the carriage actuator 516 in the direction shown by the arrow JJ in FIG. 6. This causes the outer elongate member 530 to similarly rotate. The notch 542 of the distal end portion 541 of the outer elongate member 530 contacts the pivot rod 595, causing the carriage to rotate in the direction shown by the arrow LL in FIG. 7 and into the second configuration.

Once the carriage 572 is in the second configuration, the medical tool 500 can safely be removed from the body of the patient. Said another way, once the first rotatable member 567 and the second rotatable member 568 are disposed within the first lumen 580 of the inner elongate member 550, the cutting surfaces 552, 553 cannot contact and/or damage body tissue as the medical tool is removed from the body of the patient.

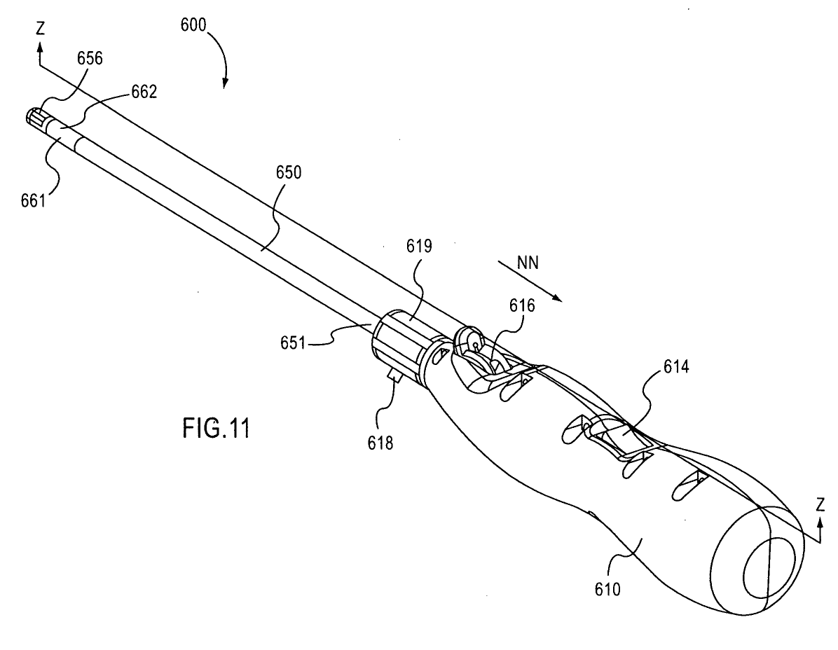

FIGS. 11-15 show a medical tool 600, according to another embodiment. Medical tool 600 is similar to medical tool 500 and includes a housing 610, an elongate member 650, a tissue disrupter 656, a threaded member 685, a flexible shaft 690, a steering rod 695 and a distal cap 662. Elongate member 650, threaded member 685, flexible shaft 690 and distal cap 662 of the medical tool 600 are similar to inner elongate member 550, threaded member 585, flexible shaft 590 and distal cap 562 of the medical tool 500, respectively. As such, the elongate member 650, the threaded member 685, the flexible shaft 690 and the distal cap 662 of the medical tool 600 are not described in detail herein.

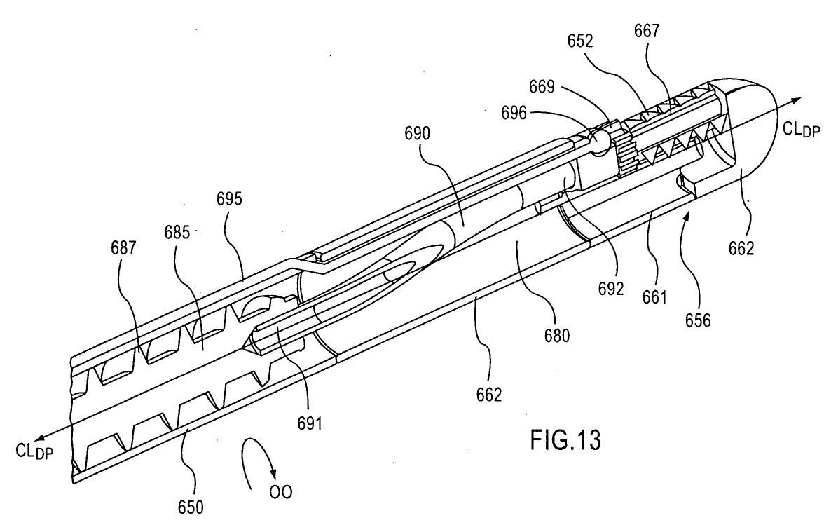

The housing 610 includes an actuation switch 614, a steering actuator 616, a motor (not shown), a battery (not shown), an optional suction port 618, and a collection vessel 619. The motor of the housing 610 is disposed within the housing 610 and is configured to be powered by the battery. The motor is coupled to the threaded member 685 and is configured to rotate the threaded member 685 in the direction shown by the arrow OO in FIG. 13 when actuated.

Similar to the actuation lever 514 of the housing 510 of the medical tool 500, the actuation switch 614 of the housing 610 is configured to actuate the tissue disrupter 656 of the medical tool 600. The actuation switch 614 is an electronic switch configured to move between an on position and an off position. When the actuation switch 614 is in its on position, the motor (not shown) is actuated. Actuation of the motor causes the threaded member 685 to rotate in the direction shown by the arrow OO in FIG. 13. Similar to the medical tool 500, rotation of the threaded member 685 causes the flexible shaft 690 and a first rotatable member 667 to rotate in a similar direction as the threaded member 685 and a second rotatable member 668 to rotate in an opposite direction. When the actuation switch 614 is in its off position, the threaded member 685 does not rotate. When the threaded member 685 does not rotate, the flexible shaft 690, the first rotatable member 667 and the second rotatable member 668 do not rotate. Said another way, moving the actuation switch 614 from its off position to its on position actuates the tissue disrupter 656.

The collection vessel 619 is coupled to the proximal end portion 651 of the elongate member 650 and is configured to receive tissue. Once the tissue collected at the distal end portion 661 of the elongate member 650 reaches the proximal end portion 651 of the elongate member 650, it is deposited into the collection vessel 619. The optional suction port 618 is configured to receive a suction source (not shown). The suction source is configured to help draw body tissue through the lumen 680 defined by the elongate member 650 from the distal end portion 661 of the elongate member 650 to the proximal end portion 651 of the elongate member 650 and into the collection vessel 619.

In some embodiments, the collection vessel includes a one-way valve, such as a pressure relief valve, configured to allow for air to escape from within the collection vessel. For example, in some embodiments, as tissue fragments are drawn into the collection vessel, air within the collection vessel may become pressurized. A pressure relief valve can be used to allow for a one-way flow of air to exit the collection vessel as tissue is moved into the collection vessel.

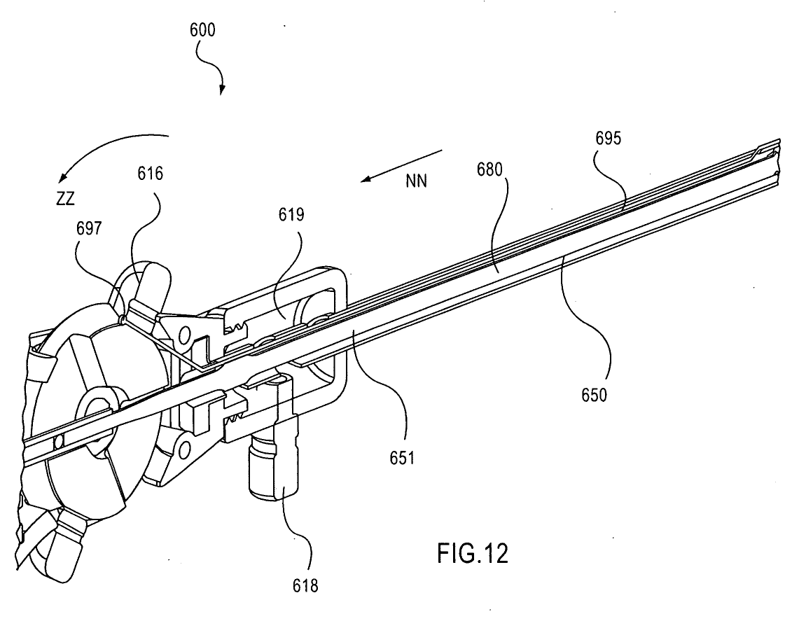

The steering actuator 616 has a first position, and a second position and is coupled to a proximal end portion 697 of the steering rod 695. A user can move the steering actuator 616 from its first position to its second position by moving the steering actuator 616 in the direction shown by the arrow ZZ in FIG. 12. Similarly, a user can move the steering actuator 616 from its second position to its first position by moving the steering actuator 616 in the direction opposite the direction shown by the arrow ZZ in FIG. 12.

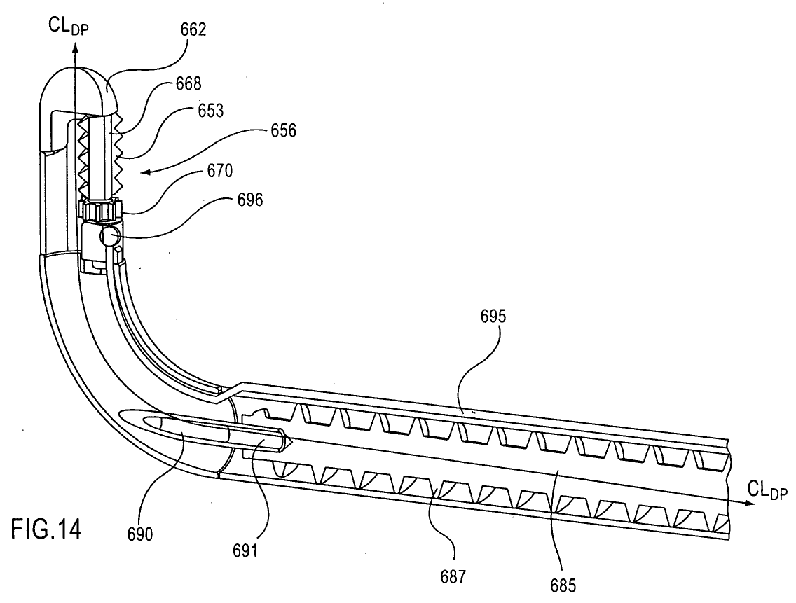

The steering actuator 616 is configured to move the distal end portion 661 of the elongate member 650 between a first position and a second position, as further described herein. Said another way, when the steering actuator 616 is in its first position, the distal end portion 661 of the elongate member 650 is in its first position (FIG. 13); when the steering actuator 616 is in its second position, the distal end portion 661 of the elongate member 650 is in its second position (FIG. 14).

The steering rod 695 has a proximal end portion 697 and a distal end portion 696. As previously stated, the proximal end portion 697 of the steering rod 695 is coupled to the steering actuator 616. A portion of the steering rod 695 is disposed within the elongate member 650. The distal end portion 696 of the steering rod 695 is coupled to the distal end portion 661 of the elongate member 650.

When the steering actuator 616 is moved from its first position to its second position, as described above, the steering rod 695 is moved in the direction shown by the arrow NN in FIG. 12. This causes a flexible portion 662 of the distal end portion 661 of the elongate member 650 to flex. When the flexible portion 662 flexes, the distal end portion 661 moves from its first position to its second position, as further described herein.

The elongate member 650 of medical tool 600 is similar to the inner elongate member 550 of medical tool 500 and has a proximal end portion 651, a distal end portion 661 and defines a lumen 680. Similar to the medical tool 500, a threaded member 685 having threads 687 is disposed within the lumen. The threaded member 685 is connected to the motor and the proximal end portion 691 of the flexible shaft 690. As described above, in this manner, when the motor rotates the threaded member 685, the flexible shaft 690 rotates in a similar direction.

The distal end portion 661 of the elongate member 650 includes a flexible portion 662. The flexible portion 662 is configured to move the distal end portion 661 of the elongate member 650 from a first position (FIG. 13) to a second position (FIG. 14). When the distal end portion 661 is in its first configuration, a center line CL_{DP} defined by the distal end portion 661 of the elongate member 650 is substantially linear. When the distal end portion 661 is in its second configuration, the center line CL_{DP} defined by the distal end portion 661 can be non-linear. Said another way, the distal end portion 661 is curved when in its second configuration.

As described above, the distal end portion 696 of the steering rod 695 is coupled to the distal end portion 661 of the elongate member 650. When the steering rod 695 is pulled in the direction shown by the arrow NN in FIG. 12, the flexible portion is configured to flex. The flexing of the flexible portion causes the distal end portion 661 to move from its first position to its second position. When the distal end portion 661 is in its second configuration, the tissue disrupter 656 can disrupt tissue that is hard to reach and/or cannot be reached when the tissue disrupter 656 is its first configuration. For example, the tissue disrupter 656 can disrupt tissue that is located away from a longitudinal axis defined by the elongate member 650. In this manner, the tissue disrupter 656 can disrupt tissue located towards the various positions along the annular fibrous wall of an intervertebral disc increasing the amount of nucleus that can be removed from the intervertebral disc.

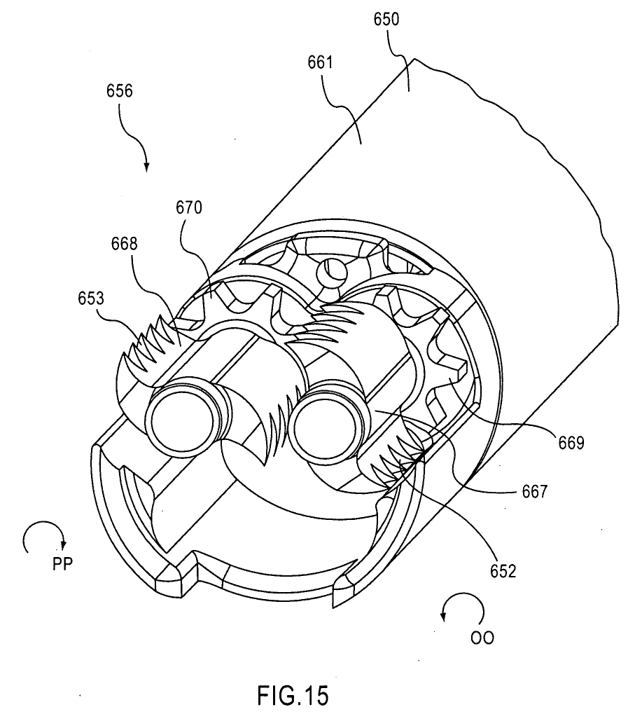

The tissue disrupter 656 of the medical tool 600 is coupled to the distal end portion 661 of the elongate member 650 and includes a first rotatable member 667 and a second rotatable member 668. The first rotatable member 667 includes a gear 669 and a cutting surface 652. The gear 669 is configured to engage a gear 670 of the second rotatable member 668, as further described herein. The cutting surface 652 of the first rotatable member 667 has two portions shaped like claws. The claws include pointed teeth that are angled such that when the first rotatable member 667 rotates in the direction shown by the arrow OO in FIGS. 13 and 15, the pointed teeth cleave, stir, disrupt, and/or sever tissue when the medical tool 600 is inserted into a body of a patient. The cutting surface 652 of the first rotatable member 667 does not contact the cutting surface 653 of the second rotatable member 668. The first rotatable member 667 is coupled to the distal end portion 692 of the flexible shaft 690 such that when the flexible shaft 690 rotates in the direction shown by the arrow OO in FIGS. 13 and 15, the first rotatable member 667 similarly rotates.

Similar to the first rotatable member 667, the second rotatable member 668 includes a gear 670 and a cutting surface 653. The gear 670 is configured to engage a gear 669 of the first rotatable member 667, as further described herein. The cutting surface 653 of the second rotatable member 668 has two portions shaped like claws. The claws include pointed teeth that are angled such that when the second rotatable member 668 rotates in the direction shown by the arrow PP in FIG. 15, the pointed teeth cleave, stir, disrupt, and/or sever tissue when the medical tool 600 is inserted into a body of a patient. The cutting surface 653 of the second rotatable member 668 does not contact the cutting surface 652 of the first rotatable member 667.

As discussed above, the gear 669 of the first rotatable member 667 and the gear 670 of the second rotatable member 668 are configured to engage each other. Rotating the first rotatable member 667 in the direction shown by the arrow OO in FIG. 15, causes the second rotatable member 668 to rotate in the direction shown by the arrow PP in FIG. 15. Thus, only one of the first tissue disrupter 667 and the second tissue disrupter 668 needs to be moved to cause both the first tissue disrupter 667 and the second tissue disrupter 668 to move.

In use, the medical tool 600 is inserted into a body of a patient by a medical practitioner with the actuation button 614 in its off position and the steering actuator 616 in its first position. For example, a medical practitioner can insert the medical tool 600 percutaneously through a cannula into a body of a patient. Similar to the methods described above in relation to medical tool 100, a medical practitioner can gain access to the interior of an intervertebral disc of a patient and insert the medical tool 600 such that the tissue disrupter 656 is disposed within the interior of the intervertebral disc of the patient.

Once the tissue disrupter 656 of the medical tool 600 is positioned within the body of the patient, the first rotatable member 667 and the second rotatable member 668 can be rotated with respect to the distal end portion 661 of the elongate member 650 in the directions shown by the arrows OO and PP in FIG. 15, respectively. As discussed above, this is accomplished by moving the actuation switch 614 of the housing 610 from its off position to its on position. Because the medical tool 600 is driven by a motor, when the actuation switch 614 of the housing is in its on position, the first rotatable member 667 and the second rotatable member 668 continuously rotate with respect to the distal end portion 661 of the elongate member 650 in the directions shown by the arrows OO and PP in FIG. 15, respectively.

By rotating the first rotatable member 667 in the direction shown by the arrow OO in FIG. 15 and the second rotatable member 668 in the direction shown by the arrow PP in FIG. 15, the cutting surface 652 of the first rotatable member 667 and the cutting surface 653 of the second rotatable member 668 contact and cleave, stir, disrupt, and/or sever the body tissue adjacent the cutting surfaces 652, 653. For example, the cutting surface 652 of the first rotatable member 667 and/or the cutting surface 653 of the second rotatable member 668 can cleave, stir, disrupt, and/or sever at least a portion of the nucleus of an intervertebral disc when the medical tool 600 is inserted into the interior of an intervertebral disc.

Once the distal end portion 661 of the elongate member 650 is disposed within the body of a patient, the distal end portion 661 of the elongate member 650 can be moved from its first position (FIG. 13) to its second position (FIG. 14). As discussed above, this enables the tissue disrupter 656 to disrupt hard to reach tissue. As such, the tissue disrupter 656 has greater mobility and can disrupt tissue that the tissue disrupter 656 could not reach with the distal end portion 661 of the elongate member 650 in its first position. For example, a greater portion of the nucleus of an intervertebral disc can be severed and/or removed.

Once the body tissue is cleaved, stirred, disrupted, and/or severed, the body tissue can be conveyed between the first rotatable member 667 and the second rotatable member 668 and into the lumen 680 defined by the inner elongate member 650. As more tissue is deposited into the lumen 680 defined by the inner elongate member 650, the tissue begins to move in a proximal direction from the distal end portion 661 of the inner elongate member 650.

As the tissue moves in a proximal direction, the tissue contacts the threaded member 685. As described above, when the actuation switch 614 is in its on position, the threaded member 685 rotates in the direction shown by the arrow OO in FIG. 13. Said another way, the threaded member 685 simultaneously rotates with the first rotatable member 667 and the second rotatable member 668. The threads 687 of the threaded member 685 contact the tissue and are configured to move the tissue away from the distal end portion 661 of the inner elongate member 650, when the threaded member rotates in the direction shown by the arrow MM in FIG. 9. In this manner, the tissue can be conveyed from the distal end portion 661 of the elongate member 650 to the collection vessel 619 of the housing 610. Once the tissue has been removed from the body of the patient, the medical tool 600 can be removed from the body of the patient.

As discussed above, a suction source can be connected to the optional suction port 618 on the housing 610. The suction provided by the suction source is configured to assist the threaded member 685 in conveying the tissue from the distal end portion 661 of the elongate member 651 to the collection vessel 619 of the housing 610.

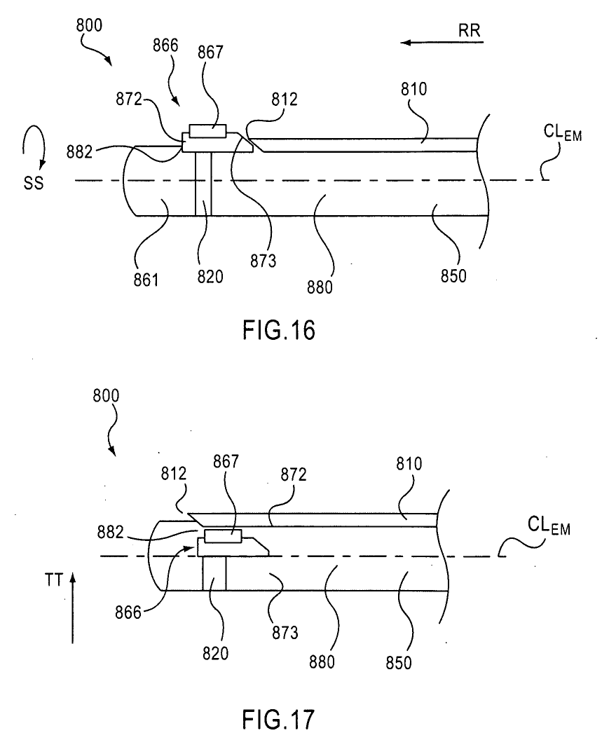

FIGS. 16 and 17 are schematic illustrations of a medical tool 800 in a first configuration and a second configuration, respectively, according to another embodiment. Medical tool 800 includes an elongate member 850, an actuation member 810, and a tissue disrupter 866. The elongate member 850 includes a distal end portion 861 and defines a lumen 880 and an aperture 882. The distal end portion 861 is configured to be inserted into a body of a patient, as further described herein. In some embodiments, the lumen 880 can be configured to receive body tissue.

The actuation member 810 slides with respect to the elongate member 850 in a direction substantially parallel to a center line CL_{EM} defined by the elongate member 850. Movement of the actuation member 810 with respect to the elongate member 850 in a direction substantially normal to the center line CL_{EM} of the elongate member 850 is limited.

The actuation member 810 includes an angled surface 812. The angled surface 812 has an angle that is supplementary to an angled surface 873 of a carriage 872 of the tissue disrupter 866. The angled surface 812 of the actuation member 810 slides along the angled surface 873 of the carriage 872, as described in further detail herein. The actuation member 810 is configured to move between a first position (FIG. 16) and a second position (FIG. 17), corresponding to the first configuration and the second configuration of the medical tool 800. When the actuation member 810 is in its first position, the tissue disrupter 866 is disposed within the aperture 882 defined by the elongate member 850. When the actuation member 810 is in its second position, the actuation member 810 is positioned such that the aperture 882 defined by the elongate member 850 is covered. Said another way, when the actuation member 810 is in its second position, the aperture 882 defined by the elongate member 850 is not in fluid communication with the area surrounding the distal end portion 861 of the elongate member 850. In this manner, the tissue disrupter 866 is entirely disposed within the lumen 880 defined by the elongate member 850 when the actuation member 810 is in its second position.

The tissue disrupter 866 of the medical tool 800 includes a carriage 872, a biasing member 820 and a rotatable member 867. The tissue disrupter 866 is coupled to the distal end portion 861 of the elongate member 850. The biasing member 820 of the tissue disrupter 866 can be, for example, a spring. The biasing member 820 has an expanded position (FIG. 16) and a compressed position (FIG. 17) corresponding to the first configuration and the second configuration of the medical tool 800, respectively. When the biasing member 820 is in its expanded position it retains the carriage 872 in a position such that the rotatable member 867 is disposed outside the lumen 880 defined by the elongate member 850. When the biasing member 820 is in its compressed position, the actuation member 810 of the elongate member 850 retains the tissue disrupter 866 within the lumen 880 defined by the elongate member 850. In the compressed position, the biasing member 820 exerts a force on the carriage 872 in the direction shown by the arrow TT in FIG. 17. This force allows the biasing member 820 to move the rotatable member 867 to a position in which the rotatable member 867 is disposed outside the lumen 880 when the actuation member 810 is moved from its second position to its first position.

The carriage 872 of the tissue disrupter 866 includes an angled surface 873. The angled surface 873 has an angle that is supplementary to the angled surface 812 of the actuation member 810. The angled surface 873 slides along the angled surface 812 of the actuation member 810 when the actuation member 810 moves with respect to the elongate member 850 in the direction shown by the arrow RR in FIG. 16.

The rotatable member 867 of the tissue disrupter 866 is coupled to the carriage 872. The rotatable member 867 is configured to rotate relative to the carriage 872 in a direction shown by the arrow SS in FIG. 16. In some embodiments, the rotatable member 867 can have a sharp edge similar to the embodiments discussed above. For example, the rotatable member 867 can include a sharp worm gear, a helical flute, and/or claws. The rotatable member 867 disrupts tissue when the rotatable member 867 of the tissue disrupter 866 is inserted into a body of a patient, as described in further detail below. In some embodiments, the rotatable member 867 of the tissue disrupter 866 can be substantially rigid. In other embodiments, the rotatable member 867 can be configured to flex and/or bend.

As shown in FIGS. 16 and 17, the medical tool 800 is movable between a first configuration and a second configuration. When the medical tool 800 is in the first configuration, the rotatable member 867 is disposed outside the lumen 880 defined by the elongate member 850. In this manner, the rotatable member can cleave, stir, disrupt, and/or sever body tissue adjacent the rotatable member 867.

When the medical tool 800 is in the second configuration, the rotatable member 867 is disposed within the lumen 880 defined by the elongate member 850. Said another way, when the medical tool 800 is in the second configuration, the rotatable member 867 is not exposed to the area surrounding the distal end portion 861 of the elongate member 850.

To move the medical tool 800 from the first configuration to the second configuration, the actuation member 810 is moved in the direction shown by the arrow RR in FIG. 16. This causes the angled surface 812 of the actuation member 810 to exert a force on the angled surface 873 of the carriage 872. Because the angled surface 812 of the actuation member 810 and the angled surface 873 of the carriage 872 are supplementary, a portion of the force exerted on the angled surface 873 of the carriage 872 is opposite the direction shown by the arrow TT in FIG. 17. This force causes the tissue disrupter 866 to compress the biasing member 820 and move the medical tool 800 from the first configuration (FIG. 16) to the second configuration (FIG. 17). When the actuation member 810 is moved in the direction opposite the direction shown by the arrow RR in FIG. 16, the biasing member 820 forces the tissue disrupter 866 through the aperture 882 defined by the elongate member 850 and moves the medical tool 800 from the second configuration to the first configuration.

In use, the medical tool 800 is inserted into a body of a patient with the medical tool 800 in the second configuration. More specifically, the tissue disrupter 866 is inserted into a body of a patient when the tissue disrupter 866 is not exposed to the area surrounding the distal end portion 861 of the elongate member 850. For example, a medical practitioner can insert the medical tool 800 percutaneously through a cannula into a body of a patient. Similar to the methods described above in relation to medical tool 100, a medical practitioner can gain access to the interior of an intervertebral disc of a patient and insert the medical tool 800 such that the tissue disrupter 866 is disposed within the interior of the intervertebral disc of the patient.

Once the medical tool 800 is positioned within the body of the patient, the medical tool 800 is moved from the second configuration to the first configuration as described above. This exposes the cutting surface 852 of the rotatable member 867 to the area surrounding the distal end portion 861 of the elongate member 850.

Once the medical tool 800 is in the first configuration, the rotatable member 867 can be rotated with respect to the carriage 872 in the direction shown by the arrow SS in FIG. 16. By rotating the rotatable member 867 in the direction shown by the arrow SS in FIG. 16, the rotatable member 867 contacts and cleaves, stirs, disrupts, and/or severs the body tissue adjacent the rotatable member 867. For example, the rotatable member 867 can cleave, stir, disrupt, and/or sever at least a portion of the nucleus of an intervertebral disc when the tissue disrupter 866 is inserted into the interior of an intervertebral disc.

Once the cutting surface 852 of the rotatable member 867 has severed the body tissue, the medical tool 800 can be removed from the body of the patient. The medical tool 800 is removed from the body of the patient by moving the medical tool 800 from the first configuration to the second configuration. As discussed above, when the medical tool 800 is in the second configuration, the rotatable member 867 is disposed within the lumen 880 defined by the elongate member 850 and does not contact the area surrounding the distal end portion 861 of the elongate member 850. Once the carriage 872 of the tissue disrupter 866 is in the second configuration, the medical tool 800 can be safely removed from the body of the patient.

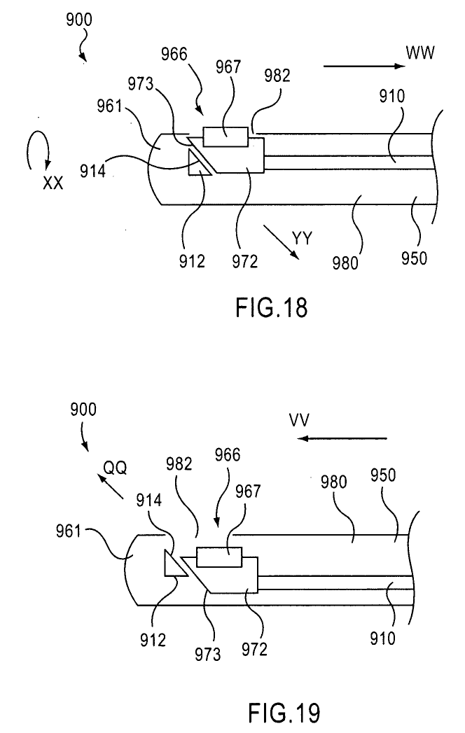

FIGS. 18 and 19 are schematic illustrations of a medical tool 900 in a first configuration and a second configuration, respectively, according to another embodiment. Medical tool 900 includes an elongate member 950, an actuation member 910, and a tissue disrupter 966. The elongate member 950 includes a distal end portion 961 and defines a lumen 980 and an aperture 982. The distal end portion 961 includes an actuation ramp 912 having an angled surface 914. The angled surface 914 of the actuation ramp 912 has a angle that is supplementary to the angle of an angled surface 973 of a carriage 972 of the tissue disruptor 966, as further described herein. The angled surface 973 of the carriage 972 slides along the angled surface 914 of the actuation ramp 912 when the medical tool 900 moves between its first configuration and its second configuration. The distal end portion 961 of the medical tool 900 is configured to be inserted into a body of a patient, as further described herein. In some embodiments the lumen 980 can be configured to receive body tissue.

In some embodiments, the angled surface 973 of the carriage 972 can be slidably coupled to the angled surface 914 of the actuation ramp 912. For example, in some embodiments, the angled surface 914 of the actuation ramp 912 can have a protrusion (not shown) with an undercut and the angled surface 973 of the carriage 972 can define a groove that corresponds to the shape of the protrusion. More particularly, the protrusion of the actuation ramp and the groove of the carriage can have trapezoidal cross-sectional shapes. In this manner, the groove of the carriage can slidingly receive the protrusion of the actuation ramp. This arrangement allows the undercut of the protrusion of the actuation ramp to slidably maintain the protrusion of the actuation ramp within the groove defined by the angled surface of the carriage. Similarly stated, in such embodiments, the groove of the angled surface of the carriage and the protrusion of the angled surface of the actuation ramp collectively allow movement of the carriage, with respect to the actuation ramp, in a direction substantially parallel to the angled surface of the actuation ramp. Moreover, the groove of the angled surface of the carriage and the protrusion of the angled surface of the actuation ramp collectively limit movement of the carriage, with respect to the actuation ramp, in a direction substantially normal to the angled surface of the actuation ramp. In some embodiments, the protrusion of the angled surface of the actuation ramp is a dovetail protrusion and the groove of the angled surface of the carriage is a dovetail groove.

The actuation member 910 is coupled to the tissue disruptor 966 and is disposed within the lumen 980 defined by the elongate member 950. The actuation member 910 is configured to move with respect to the elongate member 950 in the direction shown by the arrow WW in FIG. 18. The actuation member 910 is also configured to move with respect to the elongate member 950 in the direction shown by the arrow VV in FIG. 19. In this manner, the actuation member 910 moves the medical tool 900 between the first configuration and the second configuration as further described herein.

The tissue disrupter 966 of the medical tool 900 is movably coupled to the distal end portion 961 of the elongate member 950 and includes a carriage 972 and a rotatable member 967. As discussed above, the carriage 972 of the tissue disrupter 966 includes an angled surface 973 that has an angle that is supplementary to the angled surface 914 of the actuation ramp 912. The angled surface 973 slides along the angled surface 914 of the actuation ramp 912 when the actuation member 910 moves with respect to the elongate member 950 in the direction shown by the arrow VV in FIG. 19.

The rotatable member 967 of the tissue disrupter 966 is coupled to the carriage 972. The rotatable member 967 is configured to rotate relative to the carriage 972 in a direction shown by the arrow XX in FIG. 18. In some embodiments, the rotatable member 967 can have a sharp edge similar to the embodiments discussed above. For example, the rotatable member 967 can include a sharp worm gear, a helical flute, and/or claws. The rotatable member 967 can be configured to disrupt tissue when the rotatable member 967 of the tissue disrupter 966 is inserted into a body of a patient, as described in further detail below. In some embodiments, the rotatable member 967 of the tissue disrupter 966 can be substantially rigid. In other embodiments, the rotatable member 967 can be configured to flex and/or bend.

As shown in FIGS. 18 and 19, the medical tool 900 is movable between a first configuration (FIG. 18) and a second configuration (FIG. 19). When the medical tool 900 is in the first configuration, the tissue disrupter 966 is positioned in the aperture 982 defined by the elongate member 950 such that the rotatable member 967 is disposed outside the lumen 980 defined by the elongate member 950. When the medical tool 900 is in the first configuration, the rotatable member 967 is exposed to the area surrounding the distal end portion 961 of the elongate member 950. In this manner, the rotatable member can cleave, stir, disrupt, and/or sever body tissue adjacent the rotatable member 967.

When the medical tool 900 is in the second configuration, the rotatable member 967 is not positioned in the aperture 982 and is disposed within the lumen 980 defined by the elongate member 950. Said another way, when the medical tool 900 is in the second configuration, the rotatable member 967 is not exposed to the area surrounding the distal end portion 961 of the elongate member 950.

To move the medical tool 900 from the first configuration to the second configuration, the actuation member 910 is moved in the direction shown by the arrow WW in FIG. 18. This causes the angled surface 973 of the carriage 972 to slide along the angled surface 914 of the actuation ramp 912. In this manner, the tissue disrupter 966 moves in a direction shown by the arrow YY in FIG. 18 and into the second configuration.

To move the medical tool 900 from the second configuration to the first configuration, the actuation member 910 is moved in the direction shown by the arrow VV in FIG. 19. This causes the angled surface 914 of the actuation ramp 912 to exert a force on the angled surface 973 of the carriage 972 as the angled surface 973 of the carriage 972 slides along the angled surface 914 of the actuation ramp 912. Because the angled surface 914 of the actuation ramp 912 and the angled surface 973 of the carriage 972 are supplementary, the tissue disrupter 966 moves in the direction shown by the arrow QQ in FIG. 19 causing the medical tool 900 to move from the second configuration (FIG. 19) to the first configuration (FIG. 18).

In use, the medical tool 900 is inserted into a body of a patient with the medical tool 900 in the second configuration. More specifically, the tissue disrupter 966 is inserted into a body of a patient when the rotatable member 967 of the tissue disrupter 966 is not exposed to the area surrounding the distal end portion 961 of the elongate member 950. For example, a medical practitioner can insert the medical tool 900 percutaneously through a cannula into a body of a patient. Similar to the methods described above in relation to medical tool 100, a medical practitioner can gain access to the interior of an intervertebral disc of a patient and insert the medical tool 900 such that the tissue disrupter 966 is disposed within the interior of the intervertebral disc of the patient.

Once the medical tool 900 is positioned within the body of the patient, the medical tool 900 is moved from the second configuration to the first configuration as described above. This exposes the rotatable member 967 to the area surrounding the distal end portion 961 of the elongate member 950.

Once the carriage 972 of the tissue disrupter 966 is in the second position, the rotatable member 967 can be rotated with respect to the carriage 972 in the direction shown by the arrow XX in FIG. 18. By rotating the rotatable member 967 in the direction shown by the arrow XX in FIG. 18, the rotatable member 967 contacts and cleaves, stirs, disrupts, and/or severs the body tissue adjacent the rotatable member 967. For example, the rotatable member 967 can cleave, stir, disrupt, and/or sever at least a portion of the nucleus of an intervertebral disc when the tissue disrupter 966 is inserted into the interior of an intervertebral disc. Once the body tissue is cleaved, stirred, disrupted, and/or severed, the body tissue can be removed from the body of the patient.

Once the rotatable member 967 has severed the body tissue, the medical tool 900 can be removed from the body of the patient. The medical tool 900 is removed from the body of the patient by moving the medical tool 900 from the first configuration to the second configuration. As discussed above, when the medical tool 900 is in the second configuration, the rotatable member 967 is disposed within the lumen 980 defined by the elongate member 950 and does not contact the area surrounding the distal end portion 961 of the elongate member 950. Once the medical tool 900 is in the second configuration, the medical tool 900 can be safely removed from the body of the patient.

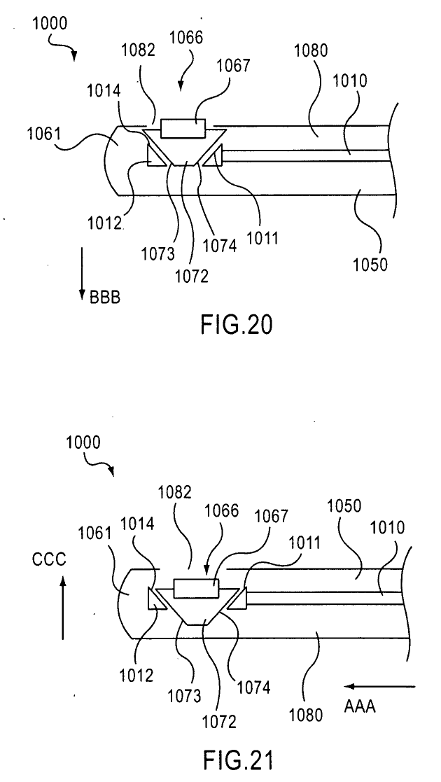

FIGS. 20 and 21 are schematic illustrations of a medical tool 1000 in a first configuration and a second configuration, respectively, according to another embodiment. Medical tool 1000 is similar to medical tool 900 and includes an elongate member 1050, an actuation member 1010, and a tissue disrupter 1066. The elongate member 1050 is similar to the elongate member 950 described above and is therefore not described in detail herein.

The actuation member 1010 includes an actuation surface 1011 and is disposed within the lumen 1080 defined by the elongate member 1050. The actuation member 1010 is configured to move with respect to the elongate member 1050 in the direction shown by the arrow AAA in FIG. 21. The actuation member 1010 is also configured to move with respect to the elongate member 1050 in the direction opposite the direction shown by the arrow AAA in FIG. 21.

The angled surface 1011 of the actuation member 1010 is angled such that it has an angle supplementary to a second angled surface 1074 of a carriage 1072 of the tissue disrupter 1066. The angled surface 1011 slides along the second angled surface 1074 of the carriage 1072. In this manner, the actuation member 1010 moves the medical tool 1000 between the first configuration and the second configuration as further described herein. In some embodiments, the angled surface 1011 of the actuation member 1010 can be slidably coupled to the second angled surface 1074 of the carriage 1072. This can be accomplished by, for example, the second angled surface of the carriage defining a groove configured to slidingly receive a protrusion of the angled surface of the actuation member. The groove of the second angled surface of the carriage and the protrusion of the angled surface of the actuation member can be similar to the protrusion and the groove described in relation to medical tool 900.

The tissue disruptor 1066 of the medical tool 1000 is movably coupled to the distal end portion 1061 of the elongate member 1050 and includes a carriage 1072 and a rotatable member 1067. The rotatable member 1067 is similar to the rotatable member 967 of the tissue disrupter 900 described above and is therefore not described in detail herein.

The carriage 1072 of the tissue disruptor 1066 includes a first angled surface 1073 and a second angled surface 1074. The first angled surface 1073 has an angle that is supplementary to an angled surface 1014 of a actuation ramp 1012. The first angled surface 1073 slides along the angled surface 1014 of the actuation ramp 1012 when the actuation member 1010 moves with respect to the elongate member 1050 in the direction shown by the arrow AAA in FIG. 21. As described above, the second angled surface 1074 has an angle that is supplementary to the angled surface 1011 of the actuation member 1010 and slides along the angled surface 1011 of the actuation member 1010. In some embodiments, the angled surface 1014 of the actuation ramp 1012 can be slidably coupled to the first angled surface 1073 of the carriage 1072 by, for example, the first angled surface of the carriage defining a groove configured to slidingly receive a protrusion of the angled surface of the actuation ramp. The groove of the first angled surface of the carriage and the protrusion of the angled surface of the actuation ramp can be similar to the protrusion and the groove described in relation with medical tool 900.

As shown in FIGS. 20 and 21, the medical tool 1000 is movable between a first configuration (FIG. 20) and a second configuration (FIG. 21). Similar to medical tool 900, the tissue disrupter 1066 is positioned in an aperture 1082 defined by the elongate member 1050 such that the rotatable member 1067 is disposed outside the lumen 1080 defined by the elongate member 1050 when the medical tool 1000 is in the first configuration. The rotatable member 1067 is disposed within the lumen 1080 defined by the elongate member 1050 when the medical tool 1000 is in the second configuration.

To move the medical tool 1000 from the first configuration to the second configuration, the actuation member 1010 is moved in the direction opposite the direction shown by the arrow AAA in FIG. 21. This causes the first angled surface 1073 of the carriage 1072 and the second angled surface 1074 to slide along the angled surface 1014 of the actuation ramp 1012 and the angled surface 1011 of the actuation member 1010, respectively. In this manner, the tissue disrupter 1066 moves in a direction shown by the arrow BBB in FIG. 20 and into the second configuration.

To move the medical tool 1000 from the second configuration (FIG. 21) to the first configuration (FIG. 20), the actuation member 1010 is moved in the direction shown by the arrow AAA in FIG. 21. This causes the angled surface 1011 of the actuation member 1010 and the angled surface 1014 of the actuation ramp 1012 to exert a force on the second angled surface 1074 of the carriage 1072 and first angled surface 1073 of the carriage 1072, respectively. A portion of this force is in the direction shown by the arrow CCC in FIG. 21. This force causes the tissue disrupter 1066 to move in the direction shown by the arrow CCC in FIG. 21 and into the first configuration (FIG. 20).

The use of the medical tool 1000 is similar to the use of the medical tool 900. As such, the use of the medical tool 1000 is not described in detail herein.

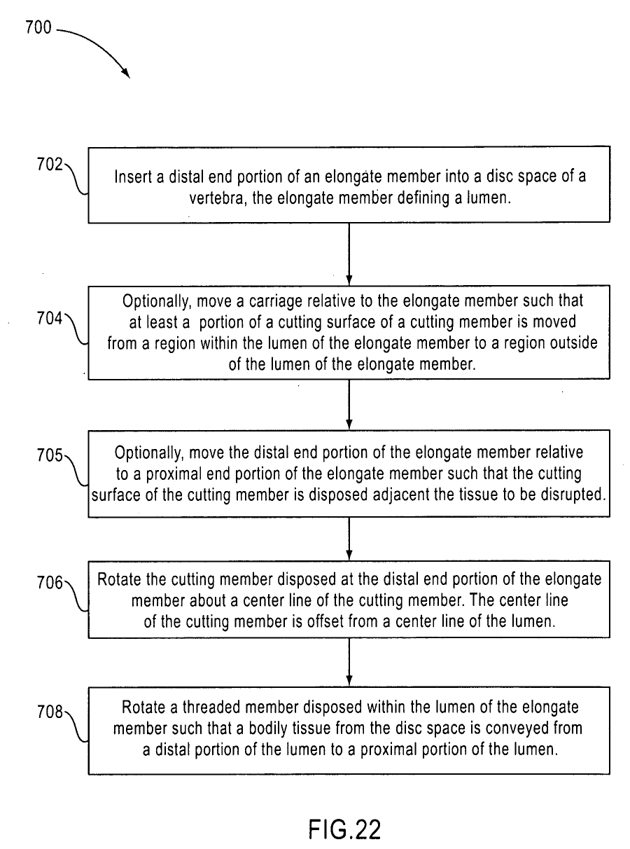

FIG. 22 is a flow chart of a method 700 of disrupting and removing tissue from a disc space of a vertebra according to an embodiment. The method 700 includes inserting a distal end portion of an elongate member into a disc space of a vertebra, at 702. The elongate member defines a lumen and can be similar to elongate members described herein. A carriage is then optionally moved relative to the elongate member such that at least a portion of a cutting surface of a cutting member is moved from a region within the lumen of the elongate member to a region outside of the lumen of the elongate member, at 704. The carriage can be similar to the carriage 572 of the medical tool 500, described above. In some embodiments, the carriage is not present and 704 is not performed.

The distal end portion of the elongate member can optionally be moved relative to a proximal end portion of the elongate member such that the cutting surface of the cutting member is disposed adjacent tissue to be disrupted, at 705. In some embodiments, this can be accomplished with a steering mechanism similar to the steering rod 695 of the medical tool 600, described above. In some embodiments, the distal end portion of the elongate member does not need to be moved and/or cannot be moved, and 705 is not performed.

A cutting member disposed at the distal end portion of the elongate member is then rotated about a center line of the cutting member, at 706. The center line of the cutting member is offset from a center line of the lumen. A threaded member disposed within the lumen of the elongate member is then rotated such that a bodily tissue from the disc space is conveyed from a distal portion of the lumen to a proximal portion of the lumen, at 708. The threaded member, can be, for example, an Archimedes screw.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Although various embodiments have been described as having particular features and/or combinations of components, other embodiments are possible having a combination of any features and/or components from any of embodiments where appropriate. For example, medical tool 600 can include a carriage similar to that of medical tool 500 and/or medical tool 500 can include a steering mechanism similar to that of medical tool 600.

What is claimed is:

1. An apparatus, comprising:
an elongate member having a distal end portion and defining a lumen; and
a tissue disruptor configured to rotate relative to the elongate member, at least a portion of the tissue disruptor being disposed within the lumen, the tissue disruptor being coupled to the distal end portion of the elongate member such that longitudinal movement of the tissue disruptor relative to the elongate member along a center line of the tissue disruptor is limited, the center line of the tissue disruptor being offset from a center line of the lumen of the elongate member.

2. The apparatus of claim 1, wherein the center line of the tissue disruptor is substantially parallel to the center line of the lumen of the elongate member.

3. The apparatus of claim 1, wherein the center line of the tissue disruptor is non-parallel to the center line of the lumen of the elongate member.

4. The apparatus of claim 1, wherein the tissue disruptor is substantially rigid.

5. The apparatus of claim 1, wherein the tissue disruptor is a first tissue disruptor configured to rotate relative to the elongate member in a first direction, the apparatus further comprising:
a second tissue disruptor configured to rotate relative to the elongate member in a second direction opposite the first direction, the second tissue disruptor coupled to the distal end portion of the elongate member.

6. The apparatus of claim 1, wherein the tissue disruptor is a first tissue disruptor, the apparatus further comprising:
a second tissue disruptor coupled to the distal end portion of the elongate member such that at least a portion of the second tissue disruptor is disposed within the lumen, the first tissue disruptor and the second tissue disruptor are configured to cooperatively macerate an object when the first tissue disruptor rotates and the second tissue disruptor rotates.

7. The apparatus of claim 1, wherein the tissue disruptor is a first tissue disruptor, an outer surface of the first tissue disruptor including a helical flute, the apparatus further comprising:
a second tissue disruptor having an outer surface including a helical flute, the second tissue disruptor coupled to the distal end portion of the elongate member such that at least a portion of the helical flute of the first tissue disruptor is engaged with at least a portion of the helical flute of the second tissue disruptor.

8. The apparatus of claim 1, wherein the tissue disruptor is a first tissue disruptor, the apparatus further comprising:
a second tissue disruptor configured to rotate relative to the elongate member, the second tissue disruptor coupled to the distal end portion of the elongate member, the first tissue disruptor and the second tissue disruptor configured to collectively lower a pressure within a space between the first tissue disruptor and the second tissue disruptor when the first tissue disruptor rotates and the second tissue disruptor rotates.

9. The apparatus of claim 1, wherein the tissue disruptor is a first tissue disruptor, the apparatus further comprising:
a second tissue disruptor configured to rotate relative to the elongate member, the second tissue disruptor coupled to the distal end portion of the elongate member, the center line of the lumen of the elongate member being offset from a plane defined by the center line of the first tissue disruptor and a center line of the second tissue disruptor.

10. The apparatus of claim 1, wherein the tissue disruptor includes a cutting surface, the apparatus further comprising:
a carriage coupled to the distal end portion of the elongate member, at least the portion of the tissue disruptor being disposed within the carriage, the carriage configured to be moved relative to the elongate member between a first position and a second position, the tissue disruptor configured such that the cutting surface is disposed within the lumen of the elongate member when the carriage is in the first position and at least a portion of the cutting surface is disposed outside of the lumen of the elongate member when the carriage is in the second position.

11. An apparatus, comprising:
an elongate member having a distal end portion and defining a lumen;
a first tissue disrupter configured to rotate relative to the elongate member in a first direction, at least a portion of the first tissue disrupter being disposed within the lumen and coupled to the distal end portion of the elongate member, a center line of the first tissue disrupter being offset from a center line of the lumen of the elongate member; and
a second tissue disrupter configured to rotate relative to the elongate member in a second direction opposite the first direction, at least a portion of the second tissue disrupter being disposed within the lumen and coupled to the distal end portion of the elongate member.

12. The apparatus of claim 11, wherein a center line of the second tissue disrupter is offset from the center line of the lumen of the elongate member.

13. The apparatus of claim 11, wherein the center line of the lumen of the elongate member is offset from a plane defined by the center line of the first tissue disrupter and a center line of the second tissue disruptor.

14. The apparatus of claim 11, wherein the center line of the first tissue disrupter is substantially parallel to the center line of the lumen of the elongate member.

15. The apparatus of claim 11, wherein the center line of the first tissue disrupter is non-parallel to the center line of the lumen of the elongate member.

16. The apparatus of claim 11, wherein the first tissue disrupter and the second tissue disrupter are configured to cooperatively macerate an object when the first tissue disrupter rotates and the second tissue disrupter rotates.

17. The apparatus of claim 11, wherein:
an outer surface of the first tissue disrupter includes a helical flute; and
an outer surface of the second tissue disrupter includes a helical flute, the second tissue disrupter coupled to the distal end portion of the elongate member such that at least a portion of the helical flute of the first tissue disrupter is engaged with at least a portion of the helical flute of the second tissue disruptor.

18. The apparatus of claim 11, wherein the first tissue disrupter is coupled to the distal end portion of the elongate member such that longitudinal movement of the first tissue disrupter relative to the elongate member along a center line of the elongate member is prevented.

19. The apparatus of claim 11, further comprising:
a carriage coupled to the distal end portion of the elongate member, at least the first tissue disrupter being movably disposed within the carriage, the carriage configured to be moved relative to the elongate member between a first position and a second position,
the first tissue disrupter configured such that a cutting surface of the first tissue disrupter is disposed within the lumen of the elongate member when the carriage is in the first position and at least a portion of the cutting surface is disposed outside of the lumen of the elongate member when the carriage is in the second position.

20. An apparatus, comprising:
an elongate member having a distal end portion and defining a lumen; and
a tissue disrupter coupled to the distal end portion of the elongate member such that movement of the tissue disrupter relative to the elongate member along a center line of the elongate member is prevented, the tissue disrupter including:
a carriage coupled to the distal end portion of the elongate member, the carriage configured to be moved between a first position and a second position; and
a rotatable member coupled to the carriage and configured to rotate relative to the carriage, the rotatable member having a cutting surface configured to be disposed within the lumen of the elongate member when the carriage is in the first position, at least a portion of the cutting surface configured to be disposed outside of the lumen of the elongate member when the carriage is in the second position.

21. The apparatus of claim 20, wherein a center line of the rotatable member is offset from a center line of the lumen of the elongate member.

22. The apparatus of claim 20, wherein the carriage is configured to rotate relative to the elongate member about a center line of the carriage, the center line of the carriage being offset from the center line of the elongate member.

23. The apparatus of claim 20, wherein:
the rotatable member is a first rotatable member configured to rotate relative to the carriage in a first direction; and
the tissue disrupter includes a second rotatable member coupled to the carriage and configured to rotate relative to the carriage in a second direction opposite the first direction.

24. An apparatus, comprising:
an elongate member having a distal end portion and defining a lumen;
a tissue disrupter coupled to the distal end portion of the elongate member, the tissue disrupter configured to convey a bodily tissue from a region outside of the elongate member into a distal portion of the lumen, the tissue disrupter configured to rotate relative to the elongate member; and
a threaded member rotatably disposed within the lumen of the elongate member, the threaded member configured to convey the bodily tissue from the distal portion of the lumen to a proximal portion of the lumen.

25. The apparatus of claim 24, wherein the tissue disrupter is coupled to the threaded member such that rotation of the threaded member relative to the elongate member results in rotation of the tissue disrupter relative to the elongate member.

26. The apparatus of claim 24, wherein the tissue disrupter is coupled to the threaded member by a flexible drive shaft such that rotation of the threaded member relative to the elongate member results in rotation of the tissue disrupter relative to the elongate member.

27. The apparatus of claim 24, wherein:
the threaded member is configured to rotate within the lumen about a center line of the threaded member; and
the tissue disrupter is configured to rotate relative to the elongate member about a center line of the tissue disruptor, the center line of the tissue disrupter being offset from and substantially parallel to the center line of the threaded member,

28. The apparatus of claim 24, wherein the tissue disrupter is a first tissue disruptor, the first tissue disrupter configured to rotate relative to the elongate member in a first direction, the apparatus further comprising:
a second tissue disrupter configured to rotate relative to the elongate member in a second direction opposite the first direction.

29. A method, comprising:
inserting a distal end portion of an elongate member into a disc space of a vertebra, the elongate member defining a lumen;
rotating a cutting member disposed at the distal end portion of the elongate member about a center line of the cutting member, the center line of the cutting member being offset from a center line of the lumen; and
rotating a threaded member disposed within the lumen of the elongate member such that a bodily tissue from the disc space is conveyed from a distal portion of the lumen to a proximal portion of the lumen.

30. The method of claim 29, wherein the inserting is performed percutaneously through a cannula.

31. The method of claim 29, wherein the cutting member is a first cutting member, the rotating the first cutting member includes rotating the first cutting member in a first direction, the method further comprising:
rotating a second cutting member disposed at the distal end portion of the elongate member in a second direction opposite the first direction and about a center line of the second cutting member.

32. The method of claim 29, further comprising:
moving a carriage, after the inserting and before the rotating the cutting member, relative to the elongate member such that at least a portion of a cutting surface of the cutting member is moved from a region within the lumen of the elongate member to a region outside of the lumen of the elongate member.
